(19) United States
(12) Patent Application Publication
(10) Pub. No.: US 2010/0081627 A1
(43) Pub. Date: Apr. 1, 2010

(54) POLYMERIC NUCLEOSIDE PRODRUGS

(75) Inventors: UmaShanker Sampath, Ballwin, MO (US); Joseph A. Toce, Webster Groves, MO (US); Sourena Nadji, Olivette, MO (US)

Correspondence Address: DARBY & DARBY P.C., P.O. BOX 770, Church Street Station, New York, NY, 10008-0770, US

(73) Assignee: RELIABLE BIOPHARMACEUTICAL, INC., St. Louis, MO (US)

(21) Appl. No.: 12/572,982

(22) Filed: Oct. 2, 2009

Related U.S. Application Data
(63) Continuation of application No. 11/668,451, filed on Jan. 29, 2007, now abandoned.
Application No. 11/668,451 is a continuation of application No. 10/739,965, filed on Dec. 17, 2003, now abandoned.
Application No. 10/739,965 is a continuation of application No. 09/853,047, filed on May 9, 2001, now abandoned.
(60) Provisional application No. 60/202,795, filed on May 9, 2000.

(51) Int. Cl. A61K 31/7052; A61P 35/00; C07H 19/10; C07H 19/20; C07H 19/06

(52) U.S. Cl. 514/47; 536/26.7; 536/26.8; 514/48; 514/49; 536/28.53; 536/28.5; 514/50

(57) ABSTRACT

Disclosed are polymeric compounds which are useful as prodrugs, comprising a chain of monomeric nucleosides, nucleoside analogs or abasic nucleosides, wherein at least one of the nucleosides or nucleoside analogs or a heterocyclic derivative thereof is pharmaceutically active and the nucleosides, nucleoside analogs or abasic nucleosides are linked by a phosphodiester group, a phosphorothioate group or an H—, alkyl or alkenyl phosphonate group.

This application claims priority under 35 U.S.C. §119 of provisional application Ser. No. 60/202,795, filed May 9, 2000.

### FIELD OF THE INVENTION

This invention is directed to the field of polymeric compounds which are useful as prodrugs. More specifically, the polymeric compounds are formed from chains of pharmaceutically active agents, particularly nucleosides and nucleoside analogs, which are linked by nuclease resistant moieties. The polymeric compounds are useful as timed release nucleoside prodrugs in the treatment of cancers, viral and microbial infections. The invention is also related to methods of treating cancer and viral and microbial infections, comprising administering the polymeric or polynucleotide compounds of the invention to a mammal in need thereof.

### BACKGROUND OF THE INVENTION

Many nucleoside compounds, nucleoside analogs or heterocyclic derivatives thereof demonstrate therapeutic activity, and a significant number of these compounds have been used as agents in treating cancers, viral infections, microbial infections and other diseases. The active agents are usually nucleoside or nucleoside analogs such as sugar modified arabino-nucleosides, base or sugar halogenated nucleosides, or a combination of base-modified nucleosides and sugar modified nucleosides or modified heterocyclic derivatives of nucleosides, such as nucleobases.

The mechanism of action of some of these nucleoside based therapeutic agents follows a unique pathway. The nucleoside is absorbed and then it is phosphorylated to the corresponding nucleoside monophosphate. (F. G. Hayden, Antimicrobial Agents: Antiviral Agents, p. 1191, and P. Calabresi, B. A. Chabner, Chemotherapy of Neoplastic Diseases, p. 1225 in The Pharmacological Basis of Therapeutics, 9th Edition, Ed. J. G. Hardman, L. E. Limbird, P. B. Molinoff, R. W. Ruddon, A. G. Gilman, et al. (1995), McGraw-Hill, New York, N.Y.). The monophosphate is then converted into the triphosphate which then terminates DNA synthesis or inhibits key enzymes required for viral replication or for cancerous cell growth. In the case of modified heterocyclic groups, such as nucleobases, there is the additional step of glycosylation prior to phosphorylation.

However, there are a number of drawbacks associated with the use of nucleoside or nucleoside analog (or heterocyclic derivative) based therapeutic agents. For example, only a small portion of the administered drug is activated by phosphorylation, leading to low monophosphate concentrations, and requiring administration of large doses to increase the amount of the active drug moiety. In addition, large doses when administered over short periods of time cause in vivo toxicity to build up. As a result, these agents have to be administered carefully and under supervision of a doctor and in a hospital. These steps increase the overall cost of the therapy to the patient and also inconvenience the patient in their daily activities.

New agents that eliminate the aforementioned problems and new methods to improve the efficiency of delivery of existing drugs have been investigated. Chapekar et al., Biochem. Biophys. Res. Commun. 115, 1, 137-143 (1983) presented data from a cell study where cordycepin (3′-deoxyadenosine) inhibited DNA and RNA synthesis when administered as a trimer (a polymer with three monomeric units). It was also noted that the main metabolite observed was the corresponding cordycepin monophosphate. Chapekar et al. did not report that this method worked in vivo.

Recently, Gmeiner et al. in Nucleosides & Nucleotides 18, 6-7, 1729-1730 (1999) and Nucleosides & Nucleotides 18, 8, 1789-1802 (1999) describe 5-FdUMP phosphodiester polymer as a prodrug for 5-fluoruacil, which is a nucleobase drug. Gmeiner et al. report the inhibition of thymidylate synthase (TS) by the degradation products of the 5-FdUMP phosphodiester polymer by cellular nucleases. Gmeiner et al. also reported that the corresponding all phosphorothioate polymer of 5-FdUMP does not show biological activity. The lack of biological activity is attributed to the stability of phosphorothioate linkages to nucleases. They concluded that the phosphorothioate oligomers are not degraded to the monomeric form of the drug to show biological activity.

The drawbacks to Gmeiner's method are two fold. Phosphodiester oligonucleotides degrade rapidly due to their inherent instability in a cellular matrix. This could lead to a rapid increase in the drug concentration leading to the in vivo toxicity observed earlier with large doses of the monomeric forms of the drug. Also, this method does not enable a controlled rate of release of the active metabolite.

Gmeiner et al., in U.S. Pat. No. 5,457,187, describes homopolymeric oligomeric forms of 5-fluorouridine and 5-fluorodeoxyuridine.

It is evident that there is a need in the art for an effective method of administering nucleoside or nucleoside analog or heterocyclic derivative thereof based therapeutic agents to patients, which does not require slow infusion by physicians or trained medical personnel.

It is still further evident that there is a need in the art for a programmed and/or controlled manner of administration of these agents.

It is still further evident that there is a need in the art to administer nucleoside agents safely, to avoid in vivo toxicity which sometimes occurs when large boluses of drugs are administered.

It is still further evident that there is a need in the art for increasing the in vivo concentration of intermediate nucleoside monophosphates in comparison to monomeric nucleoside compounds.

The present inventors have solved the problems described above by providing a safe and relatively inexpensive method for administering a pharmaceutically active agent, and particularly nucleoside or nucleoside analog or heterocyclic derivative thereof based active agent, to a patient in need thereof. Applicants have discovered a method of providing effective administration of nucleoside therapeutic agents. Applicants' method involves introducing in a controlled, programmed fashion or a personalized manner, effective concentrations of activated forms of a prodrug of the nucleoside or nucleoside analog based therapeutic agent. Applicants' method leads to administration of a reduced dosage, which degrades in an orderly/controlled manner over time to release the therapeutic agent over a desired time.

### SUMMARY OF THE INVENTION

The invention is directed in part to polymeric compounds which are useful as controlled release prodrugs.

In one embodiment, the invention is directed to a heteropolymeric compound comprising a chain of pharmaceutically active molecules, for example from 2 to 1000 molecules, which are linked with pharmaceutically inert or innocuous moieties, and to pharmaceutical compositions containing the heteropolymer. The heteropolymer is susceptible to degradation in vivo by cellular enzymes to the active pharmaceutical moiety or metabolite.

In particular embodiments, the invention is directed to polynucleotide compounds which are useful as timed release prodrugs. These polynucleotide compounds comprise sequences of pharmaceutically active nucleosides and nucleoside analogs separated by nuclease resistant moieties. The nuclease resistance moieties may be comprised of but not limited to resistance conferring nucleoside derivatives or modified backbones.

In certain embodiments, the heteropolymer is formed from a chain of pharmaceutically active monomeric nucleosides, nucleoside analogs, abasic nucleosides, or heterocyclic derivatives thereof, wherein the pharmaceutically active groups are linked by a phosphodiester group comprising a 3′ or 5′ terminal moiety, phosphorothioate group, or H— (hydrogen, known as H-phosphonates) or alkyl or alkenyl phosphonate group.

Suitable nucleosides include adenosine, 5-azacytidine, cladribine, cytarabine, doxifluridine, enocitabine, floxuridine, fludarabine, gemcitabine, pentostatin, brivudine, edoxudine, fiacitabine, fialuridine, ibacitabine, idoxuridine, ribavirin, trifluridine and vidarabine. Exemplary nucleoside analogs are acyclovir, valacyclovir, penciclovir, famciclovir, ganciclovir, cidofovir, adefovir, lobucavir and ribavirin. Other suitable nucleoside analogs contemplated for use in the invention include both carbacylic nucleosides and L-nucleosides.

Exemplary nucleobases include mercaptopurine, thioguanine and azathioprine.

For example, the chain of pharmaceutically active monomeric nucleosides, nucleoside analogs, abasic nucleosides, or heterocyclic derivatives thereof may be depicted as a heteropolymeric compound of formula I

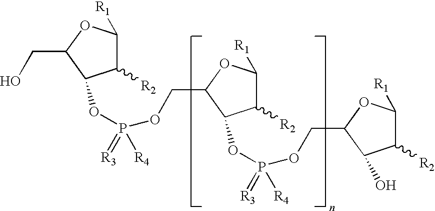

wherein R¹ is optionally present and if present is independently selected from a pharmaceutically active nucleoside, nucleoside analog or heterocyclic derivative thereof;

R² is present in the β or α face and independently selected from the group consisting of hydrogen, O—R⁵, R⁵, N—R⁵R⁶, N₃, X, or S—R⁵;

wherein R⁵ and R⁶ are independently selected from the group consisting of hydrogen, linear or branched chain alkyl, cycloalkyl, alkoxyalkyl, alkylamino, ether, thioether, haloalkyl, aryl, or heteroaryl, and wherein X is Cl, Br, F, or I; and

R³ is independently selected from the group consisting of O or S; and

n is an integer from 1-I00;

wherein when R³ is S, R⁴ is O— and when R³ is O, R⁴ is selected from the group consisting of hydrogen, alkyl, alkenyl and O⁻;

or a pharmaceutically acceptable salt thereof.

In certain embodiments of the formula (I), R¹ is selected from the group consisting of

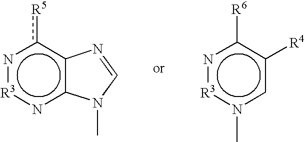

wherein R³ is CH, C═O, C═S or NH₂;

R⁴ is hydrogen, optionally substituted linear or branched alkyl, a perhalogenated alkyl, halogen and silyl;

R⁵ is O, S or NH₂; and

R⁶ is H, C═O, C═S, NH₂, NHR⁷, or SR⁷, wherein R⁷ is a linear or branched chain C₃₋₂₀ alkyl.

The invention is also directed to pharmaceutical compositions containing the heteropolymeric compounds of the invention.

In preferred embodiments, the polymeric compounds may be useful in the treatment of cancer, or in the treatment of viral or microbial infections.

### BRIEF DESCRIPTION OF THE DRAWINGS

FIG. 1 depicts a first exemplary polynucleotide prodrug of the invention;

FIG. 2 depicts a second exemplary polynucleotide prodrug of the invention;

FIG. 3 depicts a third exemplary polynucleotide prodrug of the invention;

FIG. 4 depicts a fourth exemplary polynucleotide prodrug of the invention;

FIG. 5 depicts a fifth exemplary polynucleotide prodrug of the invention;

FIG. 6 depicts a sixth exemplary polynucleotide prodrug of the invention;

FIG. 7 depicts four additional exemplary polynucleotide prodrugs of the invention;

FIG. 8 depicts four additional exemplary polynucleotide prodrugs of the invention;

FIG. 9 depicts a polynucleotide prodrug of the invention and an explanation of a timed release scenario;

FIG. 10 depicts three additional polynucleotide prodrugs of the invention and an explanation of a timed release scenario; and

FIG. 11 depicts additional exemplary heteropolymerie prodrugs of the invention.

### DETAILED DESCRIPTION OF THE INVENTION

All patents, patent applications, and references referred to herein are hereby incorporated by reference in their entirety. In the case of inconsistencies, the present disclosure, including definitions, will control.

### DEFINITIONS

As used herein, the term “nucleotide” refers to a molecule comprising a cyclic nitrogen containing base (also known as aglycone) made up of carbon, hydrogen, oxygen and nitrogen (a pyrimidine or purine), a pentose (deoxyribose, ribose, arabinose, xylose or lyxose) or hexose sugar moiety and a phosphate group (phosphorous acid).

As used herein, the term “polynucleotide” refers to a chain of nucleotide and nucleoside compounds and is used interchangeably with the term “polymers,” “oligonucleotide” and “oligo”.

As used herein, the term “oligomers” refers to oligonucleotides of progressively shorter lengths relative to the starting oligonucleotide. Oligomers may be produced in vitro and in vivo as a result of partial degradation or cleavage of the oligonucleotide.

As used herein, the term “nucleoside” refers to molecules comprising a nitrogen containing base moiety (purine or pyrimidine base) linked to a pentose (deoxyribose, ribose, arabinose, xylose or lyxose) or hexose sugar moiety. Typical purine or pyrimidine bases which form nucleosides include adenine, guanine, cytosine, 5-methyl cytosine, uracil and thymine. The term “nucleoside” as used herein includes optionally substituted nucleosides, such as nucleosides substituted with a halogen, for example, fluorine. The term “nucleoside” as used herein also includes molecules with optionally substituted heterocyclic and sugar moieties, such as substituted with a halogen, for example, fluorine.

As used herein, the term “nucleoside analog” refers to non-natural molecules or synthetically produced compounds with modifications independently or together to the sugar and base parts of the “nucleoside”, as defined above. Exemplary nucleoside analogs include acyclovir, valacyclovir, penciclovir, famciclovir, ganciclovir, cidofovir, adefovir, lobucavir and ribavirin and classes of carbacylic and L-nucleosides.

As used herein, the term “abasic nucleoside” refers to a nucleoside without a nucleobase attached at the 1′(prime)-carbon atom of the sugar moiety. The sugar hydroxyl groups of the abasic nucleoside may be variably derivatized, i.e. the hydroxyl groups may be esterified or substituted with a desired functional group or protecting group. Suitable sustituents include C₁₋₃₅ straight or branched, substituted or unsubstituted alkoxy or alkyl; C₃₋₃₅ substituted or unsubstituted cycloalkyl or cycloalkoxy; C₂₋₃₅ substituted or unsubstituted alkenyl or alkenyloxy groups; or halogens. The alkoxy, alkyl, alkenyoxy, alkenyl, cycloalkoxy or cycloalkyl groups may be substituted with one or more hydroxyl, amino or alkoxy groups, or alternatively may be substituted with one or more O, N or S atoms in the hydrocarbon chain. Preferred alkyl groups are substituted or unsubstituted C₁₋₂₀ alkyl groups, more preferably C₁₋₁₂ alkyl groups such as substituted or unsubstituted methyl, ethyl and isopropyl. Preferred alkoxy groups are substituted or unsubstituted C₁₋₂₀ alkoxy groups, more preferably C₁₋₁₂ alkoxy groups such as substituted or unsubstituted methoxy, ethoxy and isopropyloxy. Exemplary substitutents include methoxyethyl and dimethylaminoethyl. Preferred alkenyl and alkenyloxy groups have from 2 to 20 carbon atoms, more preferably from 2 to 10 carbon atoms.

As used herein, the term “heterocyclic derivative” refers to a derivative of a nucleoside or nucleoside analog. Exemplary heterocyclic derivatives include “nucleoside bases,” which are base molecules comprising a nitrogen containing base moiety (purine or pyrimidine). The term includes optionally substituted nucleoside bases, such as those substituted with a halogen, for example fluorine. Exemplary heterocyclic derivatives (for example, purine or pyrimidine derivatives) with therapeutic activity include 6-mercaptopurine, azathioprine, 5-fluorouracil and thioguanine.

As used herein, the term “pharmaceutically active agents” refers to compounds or molecules which have a demonstrated therapeutic or pharmaceutical activity, such as but not limited to antiviral, antimicrobial or anticancer activity. Suitable pharmaceutically active agents are those having polymerizable moieties, such as but not limited to hydroxyl, amino, carboxylic acid or alkenyl groups.

As used herein, the term “nucleoside therapeutic agents” refers to nucleoside or nucleoside analogs which have a demonstrated therapeutic or pharmaceutical activity, such as antiviral, antimicrobial or anticancer activity. Suitable nucleoside therapeutic agents are those having polymerizable moieties, such as hydroxyl groups. For example, a pentose moiety may be substituted with hydroxyl groups at the 3′ or 5′ positions.

Exemplary monomeric nucleoside therapeutic agents contemplated by the invention include antineoplastic agents such as adefovir, cidofivir, cladribine, (also known as leustatin), cytarabine, doxifluridine, enocitabine (also known as behenoyl cytosine arabinoside), floxuridine, fludarabine phosphate, gemcitabine, and pentostatin; and antiviral agents such as brivudine, edoxudine, fiacitabine, fialuridine, ibucitabine, idoxuridine, trifluridine, vidarabine and ribavirin.

As used herein, the term “phosphodiester” refers to the linkage —PO₄, —which is used to link the nucleoside monomers. Phosphodiester linkages (“PO”) as contemplated herein are linkages found in naturally occurring DNA. An example of nucleic acids linked by phosphodiester linkages is depicted below.

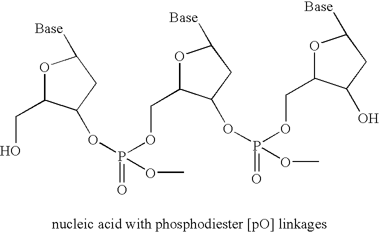

As used herein, the term “phosphorothioate” refers to the linkage —PO₃(S)— which is used to link the nucleoside monomers. Phosphorothioate (“pS”) linkages contain a sulfur atom instead of an oxygen atom on a phosphodiester linkage, as depicted below.

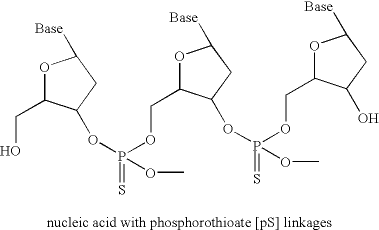

As used herein, the term “H—, alkyl or alkenyl phosphonate” refers to the linkage —PO₃R— which is used to link the nucleoside, nucleoside analog, or abasic nucleoside monomers. H-phosphonate linkages contain a hydrogen atom attached to the phosphorus instead of an oxygen atom, as in the phosphodiester linkages described above. Alkyl phosphonate linkages (R-pO) contain a carbon atom attached to the phosphorous atom instead of an oxygen atom. Suitable alkyl groups are C₁₋₃₅ linear or branched chain alkyl groups, preferably C₁₋₂₀, more preferably C₁₋₅ linear or branched alkyl. Suitable alkenyl groups are C₂₋₃₅ linear or branched chain alkenyl, preferably C₂₋₂₀, more preferably C₁₋₅ linear or branched alkenyl.

As used herein, the term “homopolymer” refers to a polynucleotide compound wherein the nucleotides and linkers are all the same. Thus, in a homopolymeric polynucleotide prodrug of the invention HO-[dN-PO₂X]ₙ—OH, each nucleotide “dN” and each X is the same.

As used herein, the term “heteropolymer” refers to a polynucleotide compound wherein each of the nucleotides or linkers in the chain are not the same. Thus, in a heteropolymeric polynucleotide prodrug of the invention HO-[dN-PO₂X]ₙ—OH, the nucleotides “dN” or the linkages “PO₂X” differ along the chain.

As used herein, the term “prodrug” refers to a molecule which is pharmaceutically inactive, but which is capable of being converted to a pharmaceutically or therapeutically active compound upon chemical or enzymatic modifications of their structure. Generally, prodrug compounds are designed to be converted to drugs in vivo.

As used herein, the term “aryl” means an aromatic carbocyclic ring system having a single radical containing 6 or more carbon atoms, and preferably from 6 to 10 carbon atoms. An aryl group may be a fused or polycyclic ring system. Exemplary aryl groups include phenyl and napthyl. The aryl groups referred to herein may be substituted with one or more substituents independently selected from the group consisting of hydroxy, protected hydroxy, cyano, nitro, alkyl, alkoxy, carboxy, protected carboxy, carbamoylmethyl, hydroxymethyl, amino, aminomethyl, trifluoromethyl, N-methylsulfonylamino, and the like.

As used herein, the term “ring system” refers to an aromatic or non-aromatic carbacyclic compound, in which one or more of the ring carbon atoms may be replaced by a heteroatom, such as nitrogen, oxygen or sulfur. The ring system may be optionally substituted by one or more substituents independently selected from the group consisting of hydroxy, protected hydroxy, cyano, nitro, alkyl, alkoxy, carboxy, protected carboxy, carbamoylmethyl, hydroxymethyl, amino, aminomethyl, trifluoromethyl, N-methylsulfonylamino, and the like.

As used herein, the term “fused ring system” refers to ring systems wherein at least two adjacent carbon centers join one or more cyclic structures. A fused ring system as used herein may be aromatic or non-aromatic, or may be composed of separate aromatic and non-aromatic moieties. Exemplary carbocyclic fused ring systems are represented by the formulae:

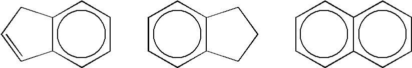

As used herein, the term “polycyclic ring system” refers to ring systems having two or more cyclic compounds bonded in tandem. A polycyclic ring system as used herein may be aromatic or non-aromatic, or may be composed of separate aromatic and non-aromatic moieties. An exemplary carbocyclic polycyclic ring system is represented by the formula

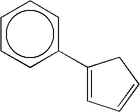

As used herein, the term “heteroaryl” means aromatic monocyclic or fused or polycyclic ring system having at least five ring atoms and a single radical, in which one or more of the atoms in the ring system is other than carbon, for example, nitrogen, oxygen or sulfur. Preferably, the heteroaryl ring has from five to ten carbon atoms. An exemplary heteroaryl group is pyridine. An exemplary fused or polycyclic heteroaryl group is indole. The heteroaryl group may be substituted by one or more substituents independently selected from the group consisting of hydroxy, protected hydroxy, cyano, nitro, allyl, alkoxy, carboxy, protected carboxy, carbamoylmethyl, hydroxymethyl, amino, aminomethyl, trifluoromethyl, N-methylsulfonylamino, and the like.

As used herein, the term “heterocycle” or “heterocyclic” means an aromatic or non-aromatic monocyclic or fused or polycyclic ring system having more than five carbon atoms, in which one or more of the atoms in the ring system is other than carbon, for example, nitrogen, oxygen or sulfur. Preferably, the heterocyclic ring has from five to ten ring atoms. A heterocycle group may be a fused or polycyclic ring system. Exemplary heterocycle groups include piperidine, morpholino and azepinyl.

As used herein, the term “alkyl” refers to a straight or branched chain alkyl moiety having from 1 to 35 carbon atoms, preferably 1 to 20, and more preferably from 1 to 12 carbon atoms. In more preferred embodiments, the alkyl group is a lower alkyl group having from 1 to 5 carbon atoms. Typical lower alkyl groups include methyl, ethyl, propyl, isopropyl, butyl, sec-butyl, t-butyl and pentyl. Alkyl groups as used herein may be optionally substituted by one or more substituents independently selected from halo, hydroxy, protected hydroxy, amino, protected amino, acyloxy, nitro, carboxy, protected carboxy, carbamyl, aryl, substituted aryl or alkoxy.

The term “haloalkyl” refers to an alkyl group which is substituted with one or more halogen groups. Exemplary haloalkyl groups include mono-substituted alkyl groups, and perhalogenated alkyl groups, such as trifluoromethyl.

The term “alkenyl” refers to a straight or branched chain hydrocarbon having a single radical and at least one carbon to carbon double bond, having from 2 to 35 carbon atoms, preferably from 2 to 20, and more preferably from 2 to 12 carbon atoms. Even more preferred alkenyl groups are lower alkenyl groups having 2 to 5 carbon atoms. The alkenyl groups referred to herein may be substituted at one or more position of the alkenyl moiety with a substituent independently selected from halo, hydroxy, protected hydroxy, amino, protected amino, acyloxy, nitro, carboxy, protected carboxy, carbamyl, aryl, substituted aryl or alkoxy.

As used herein, the term “alkynyl” as used herein includes straight chained or branched chain hydrocarbon groups having a single radical and at least one carbon to carbon triple bond, in some embodiments, having from 2 to 35 carbon atoms, preferably 2 to 20, more preferably 2 to 12 carbon atoms. More preferred alkynyl groups are those having 1 to 5 carbon atoms. The term “substituted alkynyl” as used herein refers to substitution of one or more hydrogen atoms of the alkynyl moiety with a substituent independently selected from halo, hydroxy, protected hydroxy, amino, protected amino, acyloxy, nitro, carboxy, protected carboxy, carbamyl, aryl, substituted aryl or alkoxy.

As used herein, the term “alkynyl” includes straight chained or branched chain hydrocarbon groups having a single radical and at least one carbon to carbon triple bond, in some embodiments, having from 2 to 35 carbon atoms, preferably 2 to 20, more preferably 2 to 12 carbon atoms. More preferred alkynyl groups are those having 1 to 5 carbon atoms. The term “substituted alkynyl” as used herein refers to substitution of one or more hydrogen atoms of the alkynyl moiety with a substituent independently selected from halo, hydroxy, protected hydroxy, amino, protected amino, acyloxy, nitro, carboxy, protected carboxy, carbamyl, aryl, substituted aryl or alkoxy.

As used herein, the term “cycloalkyl” refers to a cyclic alkyl group having from 3 to 25 carbon atoms, preferably from 3 to 20, and more preferably from 3 to 12 carbon atoms. Typical cycloalkyl groups include cyclopropyl, cyclopentyl and cyclohexyl. The cycloalkyl groups referred to herein may optionally be substituted with one or more substituents independently selected from halo, hydroxy, protected hydroxy, amino, protected amino, acyloxy, nitro, carboxy, protected carboxy, carbamyl, aryl, substituted aryl or alkoxy.

The term “alkoxy” is a group —OR, wherein R is a straight or branched chain alkyl group as defined above. Preferred alkoxy groups are lower alkoxy groups having from 1 to 5 carbon atoms. Exemplary preferred alkoxy groups include methoxy, ethoxy, propoxy, butoxy, sec-butoxy and pentoxy. Other exemplary alkoxy groups contemplated by the invention include heptoxy, octyloxy, and the like.

As used herein, the term “ether” refers to a group R—O—R, wherein each of the R groups are independently selected from an alkyl, alkenyl or alkynyl moiety, as defined above.

The term thioether refers to a group R—S—R, wherein each of the R groups are independently selected from an alkyl, alkenyl or alkynyl moiety, as defined above.

The term “halo” or “halogen” encompasses fluorine, chlorine, bromine and iodine.

The term “silyl” refers to a group R₃Si, wherein each of the R groups are independently selected from an alkyl, alkenyl or alkynyl moiety, as defined above.

As used herein, the term “TBDMS” refers to tertiary-butyl dimethyl silyl; the term “DMT” refers to dimethoxytrityl; the term “TBAF” refers to tetra-butyl ammonium fluoride; the term “THF” refers to tetrahydrofuran; the term “ACN” refers to acetonitrile; the term “DMF” refers to dimethylformamide; the term “Ac” refers to acetyl; the term “Et” refers to ethyl; the term “Me” refers to methyl; the term “Ph” refers to phenyl; the term “Bz” refers to benzoyl; the term “i-Pr” refers to isopropyl; and the term “TMS” refers to trimethylsilyl.

### Polymeric Compounds of the Invention

The invention is directed to polymeric compounds which are formed from a chain of pharmaceutically active molecules, which are linked by pharmaceutically inert linkages. In one embodiment, the pharmaceutically active molecules are therapeutic monomeric nucleosides, nucleoside analogs, abasic nucleosides or heterocyclic derivatives thereof which are separated along the chain by nuclease resistant moieties such as 2′O-methyl ribonucleosides. The monomeric groups may be linked along the chain by phosphodiester (pO), phosphorothionate (pS) or alkyl or alkenyl phosphonate (R-pO) groups. In a preferred embodiment, the chain comprises from 2 to 100 (more preferably from 2 to 35) therapeutic monomeric nucleosides, nucleoside analogs, abasic nucleosides or heterocyclic derivatives thereof.

In one embodiment, the invention is directed to polynucleotide compounds which are formed from a chain of pharmaceutically active or therapeutic monomeric nucleosides or nucleoside analogs which are linked by phosphodiester (pO), phosphorothionate (pS), or H—, or alkyl or alkenyl phosphonate (R-pO) groups. In a preferred embodiment, the chain comprises from 2 to 100 (more preferably 2 to 35) therapeutic monomeric nucleosides, nucleoside analogs or abasic nucleosides.

The invention contemplates polynucleotides containing mixed phosphodiester and phosphorothioate linkages, as depicted below:

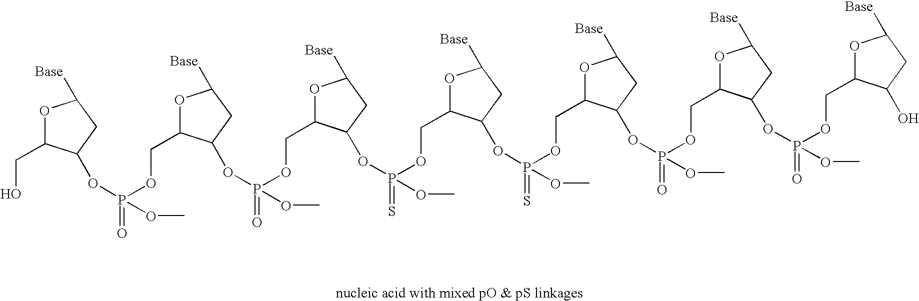

Oligomers having pS linkages are more stable than oligomers having pO linkages, and consequently pS oligomers degrade at a slower rate than pO linked oligomers S. T. Crooke, Oligonucleotide Therapeutics, in Burger's Medicinal Chemistry and Drug Discovery, 5^{th} ed., Ed. M. E. Wolff 863-900, 1995, and further references cited therein.

The invention contemplates the use of alternating chains of therapeutically active nucleosides or nucleosides analogs and nuclease resistant moieties (such as abasic 2′ O-methyl nucleosides) to form a polynucleotide containing the ability for a controlled rate of release of nucleoside therapeutic agents. The structure of alternating segments of therapeutically active nucleosides or nucleoside analogs and nuclease resistant moieties (such as abasic 2′ O-methyl nucleosides) can be modified to achieve a desired release of therapeutic agents.

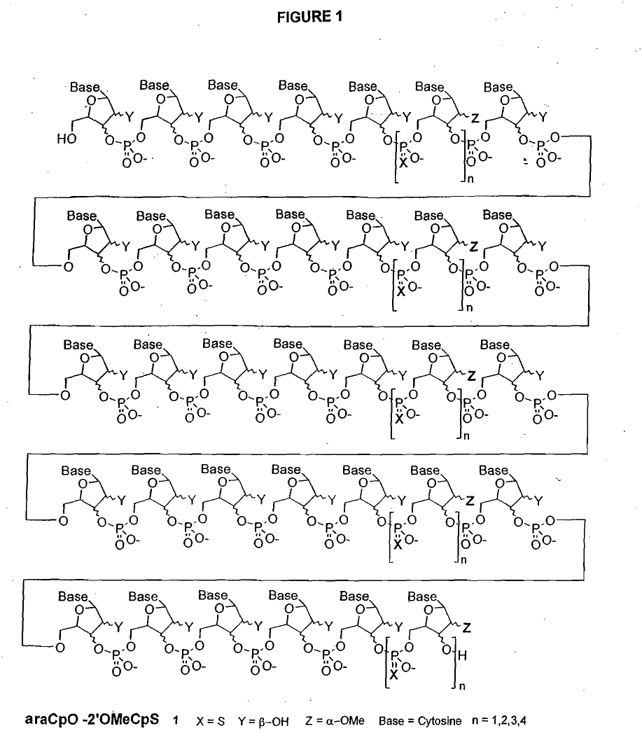

FIGS. 1-8 and 11 depict exemplary polymeric prodrugs of the invention, containing alternating segments of phosphodiester and phosphorothioate linkages or nucleobase or sugar modified nucleosides.

FIG. 1 depicts a polymeric chain of two nucleosides. The first nucleoside, the pharmaceutically active araC (cytarabine), is linked with phosphodiester linkages. The second nucleoside, 2′O-methyl-cytidine, has a low susceptibility to nucleases, and is attached with phosphorothioate linkages.

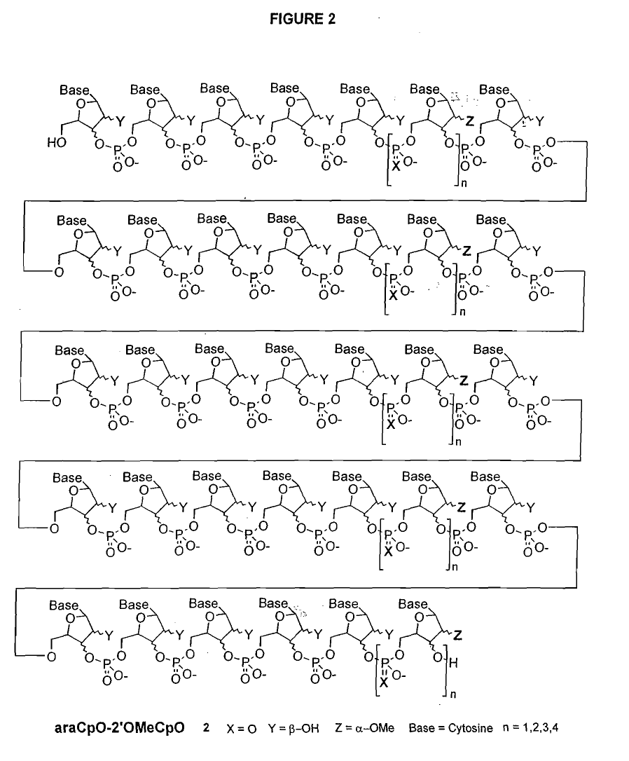

FIG. 2 depicts a polymeric chain of two nucleosides, the pharmaceutically active araC (cytarabine) and 2′O-Me-cytidine, with all phosphodiester linkages.

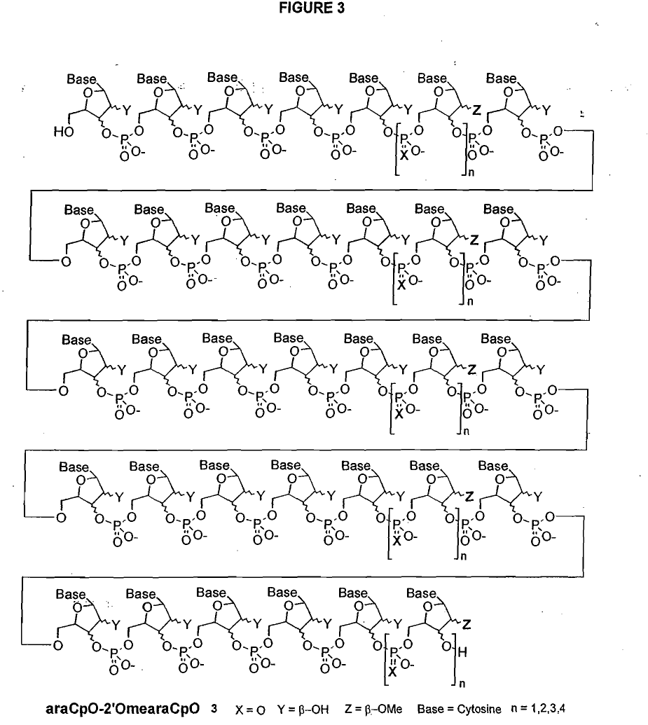

FIG. 3 depicts a polymeric chain of two nucleosides, the pharmaceutically active araC (cytarabine) and 2′O-Me-cytarabine (2′O-Me-araC), a nucleoside with very low susceptibility to nucleases and with all phosphodiester linkages.

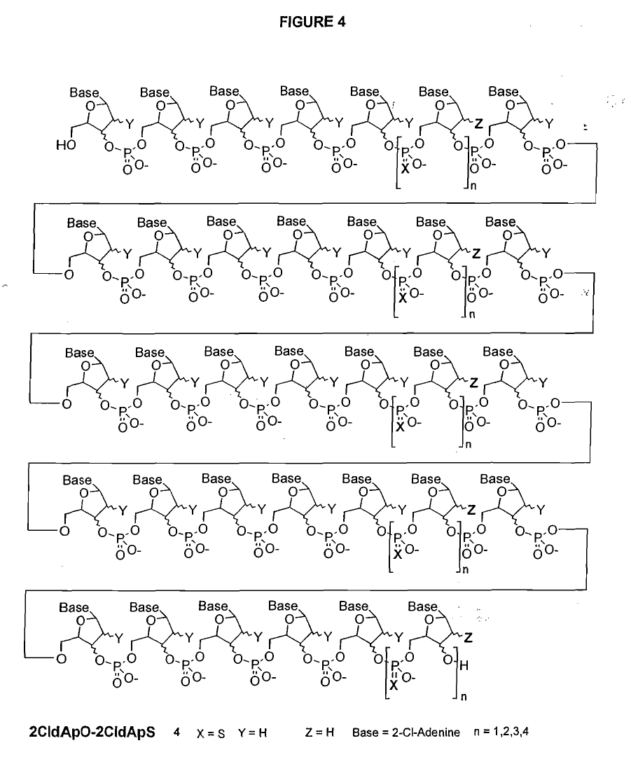

FIG. 4 depicts a polymeric chain of 2-chloro-deoxyadenosine nucleosides with groups of phosphodiester and the nuclease resistant phosphorothioate linkages.

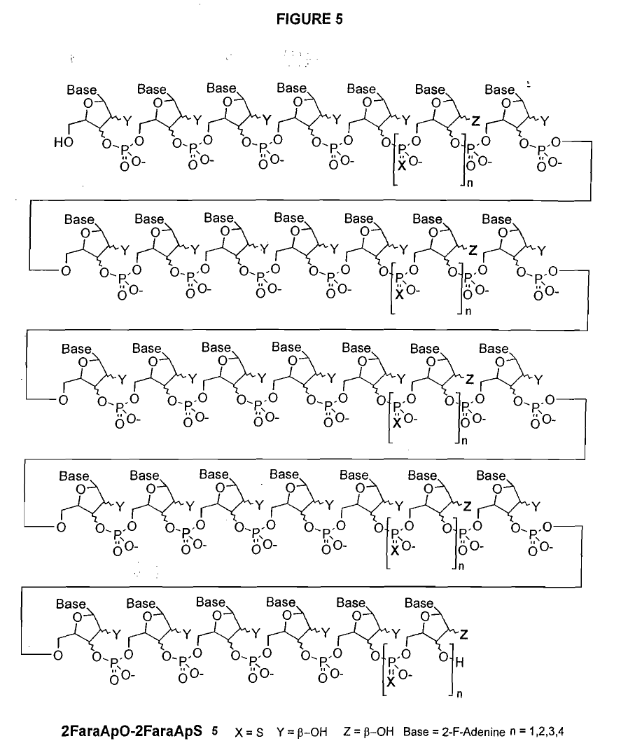

FIG. 5 depicts a polymeric chain of 2-fluoro-2′-ara-adenosine nucleosides with groups of phosphodiester and the nuclease resistant phosphorothioate linkages.

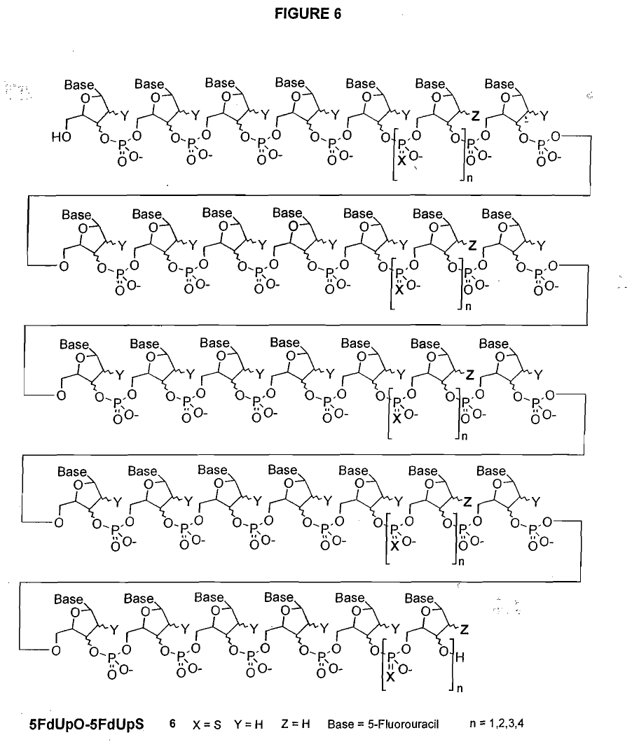

FIG. 6 depicts a polymeric chain of 5-fluoro-2′-deoxyuridine nucleosides with groups of phosphodiester and the nuclease resistant phosphorothioate linkages.

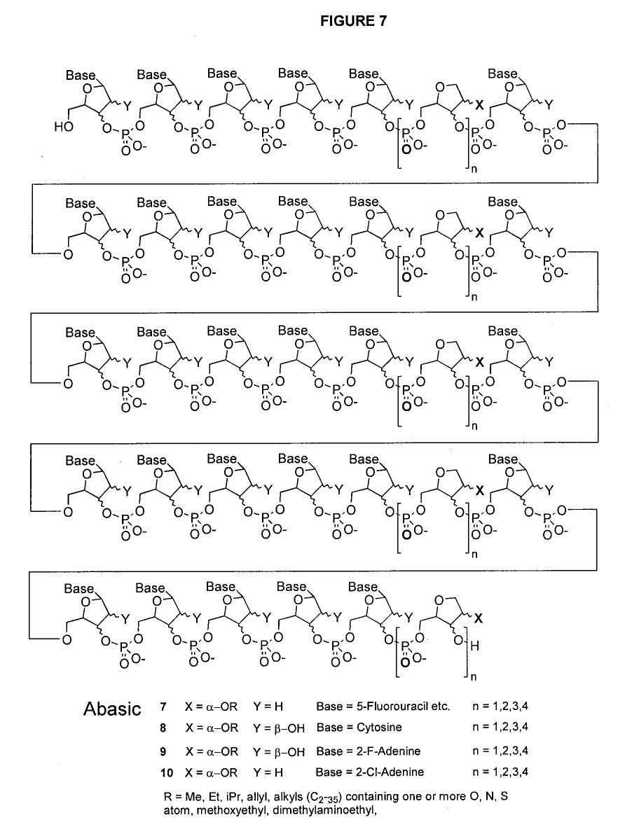

FIG. 7 depicts polymers of four different therapeutically active nucleosides or nucleoside analogs separated by abasic nucleosides. In FIG. 7, the ‘Base’ moiety chosen from 5-fluorouracil, cytosine, 2-F-adenine, or 2-Cl-adenine and the Y moiety chosen from H or β-OH determine the nature of the nucleoside, and the X moiety determines the nature of the abasic nucleoside.

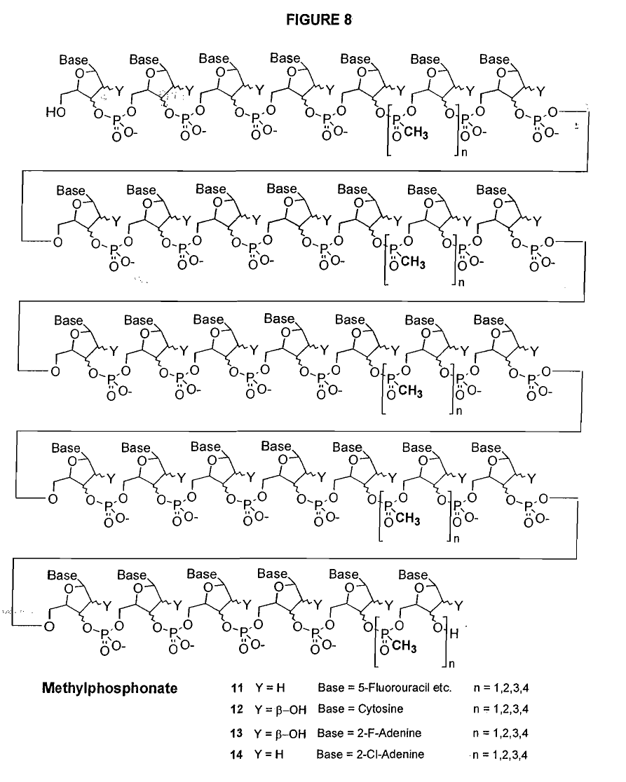

FIG. 8 depicts a polynucleotide containing mixed phosphodiestcr and alkyl phosphonate (e.g., methylphosphonate) linkages. Depending on the Y group, the nucleoside of FIG. 8 may be 5-fluorouracil, cytosine, 2-F-adenine, or 2-Cl-adenine.

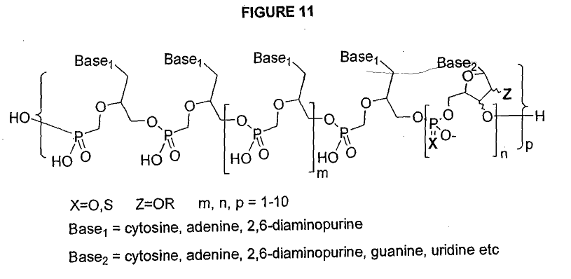

FIG. 11 depicts a polymer containing therapeutic nucleoside analog molecules and nuclease resistant nucleosides.

### NOVEL INTERMEDIATE COMPOUNDS OF THE INVENTION

The invention is also directed in part to novel compounds of the invention, which are intermediates in the synthesis of the polymeric prodrug compounds. The invention is directed to novel compounds of general formula II

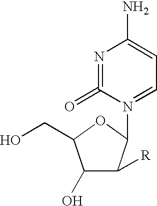

wherein R is selected from the group consisting of optionally substituted alkyl, cycloalkyl, alkoxy, alkylamino, ether, thioether, alkenyl, aryl, non-aromatic heterocyclic, and heteroaryl.

Exemplary R groups are alkoxy groups substituted with ether, such as —OCH₂OCH₂CH₃.
The invention is also directed in part to novel compounds of general formula III

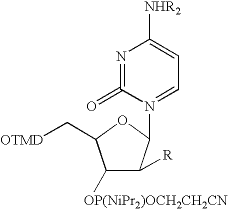

wherein R² is —C(O)R wherein R is independently selected from the group consisting of optionally substituted alkyl, cycloalkyl, alkoxy, alkylamino, ether, thioether, alkenyl, alkenyloxy, aryl, non-aromatic heterocyclic, or heteroaryl.

In particular embodiments, the R group is optionally substituted with alkoxy, for example —OCH₂OCH₂CH₃.

The invention also contemplates compounds of general formula (IV)

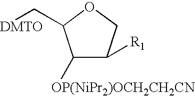

wherein R¹ is selected from the group consisting of hydrogen, optionally substituted alkyl, alkenyl, alkoxy, alkenyloxy, cycloalkyl, alkylamino, ether, thioether, aryl, non-aromatic heterocyclic, or heteroaryl. In particular embodiments, R is an optionally substituted alkyl (for example unsubstituted ethyl or methyl), or alkoxy, for example alkoxy substituted with alkoxy, such as —OCH₂OCH₂CH₃.

The invention also contemplates compounds in which the compounds of formulae II, III and IV are appended singly or as multimers or as groups of multimers to an oligonucleotide or analog at the 3′-, 5′- or at both termini.

The polymeric prodrugs of the invention allow for a preferred mode of action of the corresponding pharmaceutically active nucleoside base. In prior art methods, the pharmaceutically active nucleoside base is glycosylated in vivo to its corresponding nucleoside. The nucleoside is then converted to its monophosphate, which may be further converted to the triphosphate, as depicted below.

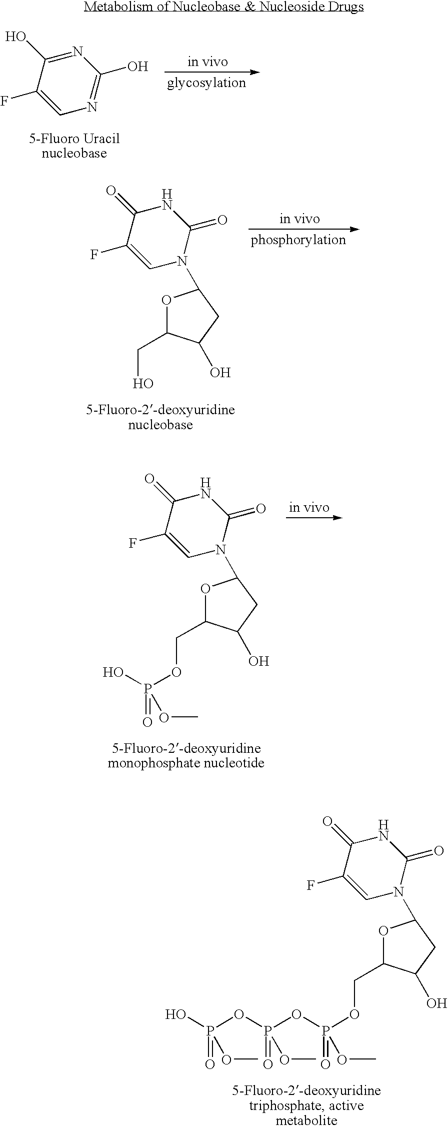

Use of the polymeric (polynucleotide) prodrug allows avoiding the reaction steps required by prior art methods using pharmaceutically active nucleoside and nucleoside analogs or heterocyclic derivatives thereof. The polynucleotide prodrug is hydrolyzed in vivo to the nucleoside or its monophosphate, which eliminates the difficult and low yielding glycosylation and phosphorylation steps. The hydrolysis of the polynucleoside prodrug of the invention in vivo is depicted below:

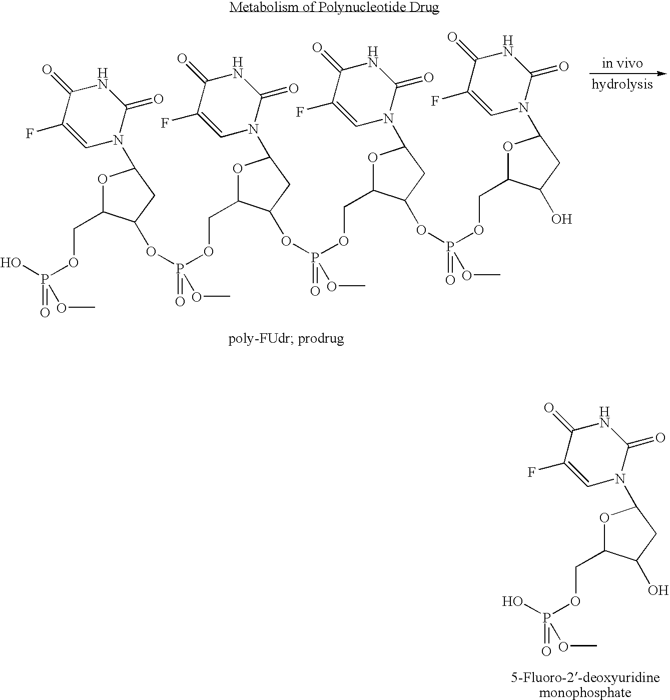

Intracellular degradation of polynucleotides is well known to those of ordinary skill in the art, and is taught by such references as S. T. Crooke, Oligonucleotide Therapeutics, in Burger's Medicinal Chemistry and Drug Discovery, 5^{th} ed., Ed. M. E. Wolff, 863-900, 1995, and further references cited therein. It is well established that oligomers with all pS linkages are more stable than oligomers with all pO linkages. Degradation occurs primarily by nuclease, through the 3′ exonuclease activity. All pO oligomers are easily degraded by cellular nucleases. All pS oligomers are very stable in cells, cell extracts, serum, urine, and are resistant to nucleases. However, pS endcapped oligomers have been degraded by nucleases faster than all pS oligomers. See, e.g., G. D. Hoke et al, Nuc. Acids. Res. 199 (20), 5743-5748, 1991.

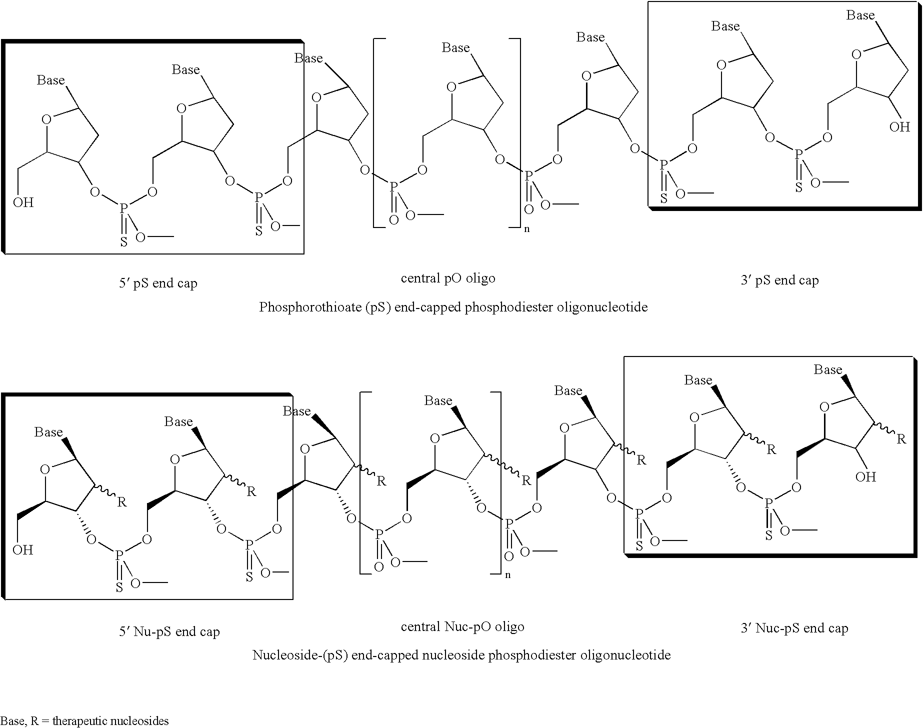

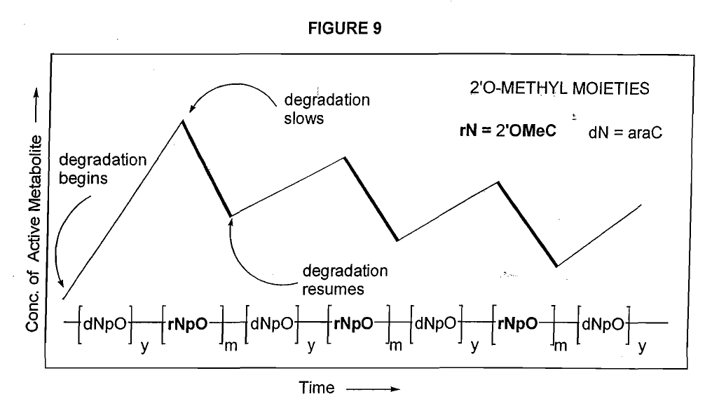

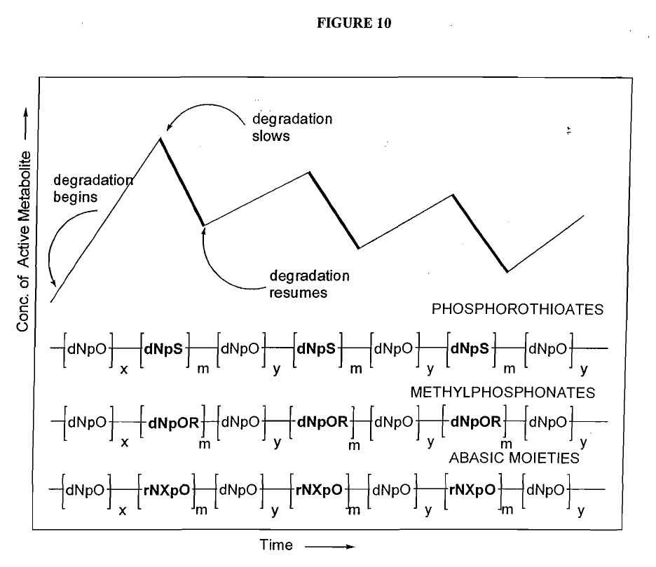

The invention contemplates the use of alternating pO and pS linkages to form a polynucleotide containing the ability for a controlled rate of release of nucleoside therapeutic agents as in FIGS. 4-6. The structure of alternating pO or pS linkages can be modified to achieve a desired release of nucleoside therapeutic agents. An explanation of the timed release and degradation scenario is depicted in FIGS. 9 and 10.

Referring to FIGS. 4-6, polynucleotide prodrugs containing a mixed phosphodiester and phosphorothioate backbone can be used as timed release drugs. Since phosphodiester groups are degraded to the corresponding nucleoside or its monophosphates faster than the corresponding phosphorothioate groups, the placement of phosphorothioate groups along a predominantly phosphodiester polynucleotide (for example, according to the pattern dNpS-dNpO-dNpO-dNpO-dNpS-dNpO-dNpO-dNpO- . . . ) will allow for release of a portion of the active drug metabolite, then act as a “speed bump” to cause slowing down of the release because of the presence of the harder to hydrolyze bonds of the phosphorothioate groups. After cleavage of the phosphorothioate groups, the second segment of phosphodiester linked nucleosides will be cleaved to allow release of the second bolus of active drug metabolite. The pattern will continue until all of the polynucleotide is degraded. The polynucleotide prodrug or nucleoside monophosphorothioate may also function as a drug or may be cleared from the system without further effects. An explanation of the timed release of the embodiments described above are depicted in FIG. 10.

The invention also contemplates the use of alternating groups of pO and R-pO linkages to form a polynucleotide containing the ability for a controlled rate of release of nucleoside therapeutic agents as shown in FIG. 8. The structure of alternating segments of pO and R-pO linkages can be modified to achieve a desired release of therapeutic agents.

The invention contemplates the use of alternating groups of therapeutically active nucleosides, nucleoside analogs with nuclease resistant moieties with suitably chosen pO or pS linkages to form a polynucleotide containing the ability for a controlled rate of release of nucleoside therapeutic agents as depicted in FIGS. 1-3. The structure of alternating segments of nucleosides and linkages can be modified to achieve a desired release of therapeutic agents. An explanation of the timed release of the embodiments described above are depicted in FIG. 9.

The invention contemplates the use of alternating groups of therapeutically active nucleoside analogs with nuclease resistant moieties with suitably chosen linkages to form a polymer containing the ability for a controlled rate of release of nucleoside therapeutic agents as depicted in FIG. 11. The structure of alternating segments of nucleoside analogs and linkages can be modified to achieve a desired release of therapeutic agents.

The invention contemplates the use of alternating chains of therapeutically active nucleosides, nucleoside analogs and nuclease resistant ‘abasic 2′-O-methyl nucleosides’ to form a polynucleotide containing the ability for a controlled rate of release of nucleoside therapeutic agents as shown in FIG. 7. The structure of alternating segments of therapeutically active nucleosides or nucleoside analogs and nuclease resistant ‘abasic 2′-O-methyl nucleosides’ can be modified to achieve a desired release of therapeutic agents. An explanation of the timed release of the embodiments described above are depicted in FIG. 9.

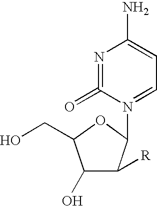

wherein R is selected from the group consisting of optionally substituted alkyl, cycloalkyl, alkoxy, alkylamino, ether, thioether, alkenyl, aryl, non-aromatic heterocyclic, and heteroaryl.

Exemplary R groups are alkoxy groups substituted with ether, such as —OCH₂OCH₂CH₃.

### Pharmaceutical Compositions

The present invention also encompasses all pharmaceutically acceptable salts of the foregoing compounds. One skilled in the art will recognize that acid addition salts of the presently claimed compounds may be prepared by reaction of the compounds with the appropriate acid via a variety of known methods. Alternatively, alkali and alkaline earth metal salts are prepared by reaction of the compounds of the invention with the appropriate base via a variety of known methods. For example, the sodium salt of the compounds of the invention can be prepared via reacting the compound with sodium hydride.

The invention contemplates the use of the compounds in various pharmaceutical forms.

Various oral dosage forms can be used, including such solid forms as tablets, gelcaps, capsules, caplets, granules, lozenges and bulk powders and liquid forms such as emulsions, solutions and suspensions. The compounds of the present invention can be administered alone or can be combined with various pharmaceutically acceptable carriers and excipients known to those skilled in the art, including but not limited to diluents, suspending agents, solubilizers, binders, retardants, disintegrants, preservatives, coloring agents, lubricants and the like.

When the compounds of the present invention are incorporated into oral tablets, such tablets can be compressed, tablet triturates, enteric-coated, sugar-coated, film-coated, multiply compressed or multiply layered. Liquid oral dosage forms include aqueous and nonaqueous solutions, emulsions, suspensions, and solutions and/or suspensions reconstituted from non-effervescent granules, containing suitable solvents, preservatives, emulsifying agents, suspending agents, diluents, sweeteners, coloring agents, and flavoring agents. When the compounds of the present invention are to be injected parenterally, they may be, e.g., in the form of an isotonic sterile solution. Alternatively, when the compounds of the present invention are to be inhaled, they may be formulated into a dry aerosol or may be formulated into an aqueous or partially aqueous solution.

In addition, when the compounds of the present invention are incorporated into oral dosage forms, it is contemplated that such dosage forms may provide an immediate release of the compound in the gastrointestinal tract, or alternatively may provide a controlled and/or sustained release through the gastrointestinal tract. A wide variety of controlled and/or sustained release formulations are well known to those skilled in the art, and are contemplated for use in connection with the formulations of the present invention. The controlled and/or sustained release may be provided by, e.g., a coating on the oral dosage form or by incorporating the compound(s) of the invention into a controlled and/or sustained release matrix.

Specific examples of pharmaceutically acceptable carriers and excipients that may be used to formulate oral dosage forms, are described in the Handbook of Pharmaceutical Excipients, American Pharmaceutical Association (1986). Techniques and compositions for making solid oral dosage forms are described in Pharmaceutical Dosage Forms: Tablets, Ed. Lieberman et al., 2nd ed., published by Marcel Dekker, Inc. Techniques and compositions for making tablets (compressed and molded), capsules (hard and soft gelatin) and pills are also described in Remington's Pharmaceutical Sciences, Ed. A. Osol, 1553-1593 (1980). Techniques and composition for making liquid oral dosage forms are described in Pharmaceutical Dosage Forms: Disperse Systems, Ed. Lieberman et al., published by Marcel Dekker, Inc.

When the compounds of the present invention are incorporated for parenteral administration by injection (e.g., continuous infusion or bolus injection), the formulation for parenteral administration may be in the form of suspensions, solutions, emulsions in oily or aqueous vehicles, and such formulations may further comprise pharmaceutically necessary additives such as stabilizing agents, suspending agents, dispersing agents, and the like. The compounds of the invention may also be in the form of a powder for reconstitution as an injectable formulation.

Preferred dosages of the compounds of the present invention are dependent upon the affliction to be treated, the severity of the symptoms, the route of administration, the frequency of the dosage interval, the presence of any deleterious side-effects, and the particular compound used, among other things.

Polynucleotide compounds of the invention can be polymerized by various methods known in the art. For example, the polynucleotide compounds can be polymerized by chemical methods using phosphoramidites, H-phosphonates or mono or triphosphates, and by way of enzymes, using triphosphates.

The polynucleotide compounds may be terminal derivatized (i.e., may be esterified or substituted with a desired functional group or protecting group) or formulated to increase cellular uptake. Methods of derivatizing or formulating the polymeric compounds will be known to those of ordinary skill in the art in view of this disclosure.

### Exemplary Embodiments of the Invention

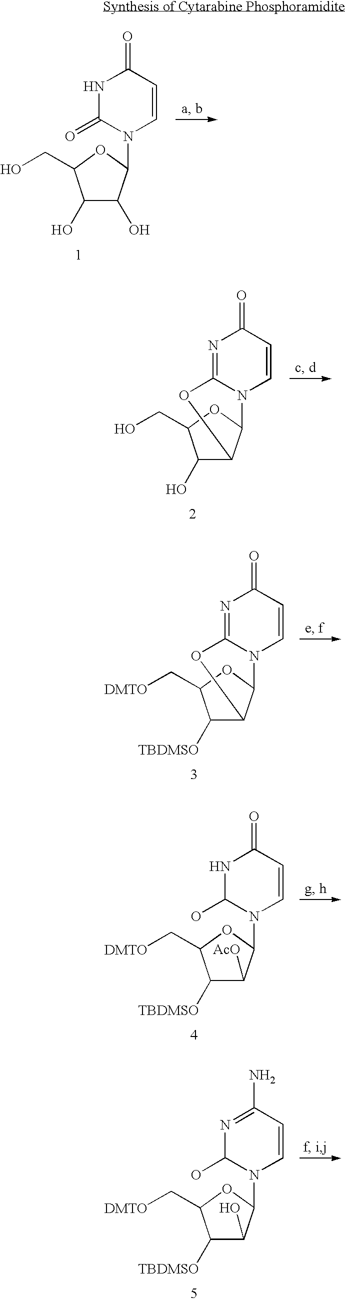

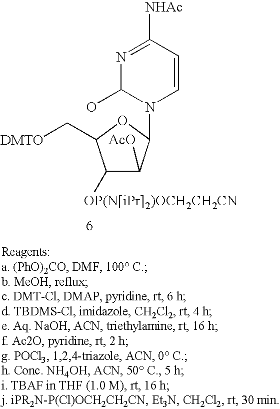

The anticancer drug cytarabine was prepared according to the procedures illustrated below. 2,2′-Anhydrouridine, 2 is available in commercial quantities from Reliable Biopharmaceutical Corporation (St. Louis, Mo.) and prepared from uridine, 1 following the standard methods shown above.

### Example 1

### Synthesis of Protected-Anhydrouridine 3

The 2,2′-anhydrouridine, 2 (87 g, 0.385 mol, 1.0 eq) was coevaporated with pyridine (2×500 mL). The residue was suspended in pyridine (3000 mL) and to it was added 4-dimethylaminopyridine (DMAP, 4.7 g, 38.5 mmol, 0.1 eq) and dimethoxytrityl chloride (DMT-Cl, 156 g, 0.46 mole, 1.2 eq). The reaction mixture was stirred for 6 h at room temperature. Thin layer chromatography (TLC, SiO₂, 3:7 MeOH:EtOAc) monitoring showed complete reaction. The reaction mixture was quenched with methanol (MeOH, 80 mL) and then evaporated to remove pyridine. The residue was coevaporated with toluene and the residue dissolved with ethyl acetate (EtOAc) and extracted with water. The extract was concentrated and the crude product purified by flash silica gel column chromatography to give the 5′-DMT-2,2′-anhydrocytidine (106 g, 52%). The product identity was confirmed by ¹H NMR (300 MHz, DMSO-d₆, δ ppm) 7.95 (1H, d, J=7.5), 7.2 (9H, m), 6.8 (4H, m), 6.32 (1H, d, J=5.6), 5.97 (1H, d, J=4.6), 5.88 (1H, d, J=6.6), 5.21 (1H, m), 4.3 (1H, m), 4.22 (1H, m), 3.73 (6H, s), 3.34 (1H, s, H₂O), 2.94 (1H, m), 2.81 (1H, m), 2.1 (1H, s, Acetone).

The 5′-DMT-2,2′-anhydrouridine (110 g, 208 mmol, 1.0 eq) was dissolved in dichloromethane (CH₂Cl₂, 3000 mL) and treated with imidazole (28 g, 416 mmol, 2.0 eq) and then tert.-butyldimethylsilyl chloride (TBDMS-Cl, 43 g, 291 mmol, 1.4 eq). The reaction mixture was extracted with aqueous NaHCO₃, dried and concentrated on a rotary evaporator. The crude product was purified by flash silica gel column chromatography with EtOAc, 1:9 MeOH:EtOAc and 2:8 MeOH:EtOAc to give the 5′-DMT-3′-TBDMS-2,2′-anhydrouridine, 3 (140 g). The product identity was confirmed by ¹H NMR (300 MHz, DMSO-d₆, δ ppm) 7.94 (1H, d, J=7.5), 7.2 (9H, m), 6.84 (4H, m), 6.34 (1H, d, J=5.9), 5.88 (1H, d, J=7.5), 5.25 (1H, m), 4.5 (1H, m), 4.1 (1H, m), 3.72 (6H, s), 3.34 (1H, s, H₂O), 3.06 (1H, dd, J=11.6, 4.4), 2.85 (1H, dd, J=10.6, 5.7), 0.84 (9H, s), 0.69 (3H, s), −0.015 (3H, s).

### Example 2

### Synthesis of Protected-Arabinouridine 4

The anhydro moiety of the intermediate, 3 was opened by reaction with triethylamine. The 5′-DMT-3′-TBDMS-2,2′-anhydrouridine, 3 (140 g, 218 mmol) was dissolved in acetonitrile (ACN, 500 mL) and to it was added triethylamine (250 mL) and aqueous sodium hydroxide (NaOH, 0.25 M, 250 mL). The reaction mixture was stirred for 16 h at room temperature. TLC (SiO₂, 1:9 MeOH:EtOAc) monitoring showed complete reaction. The reaction mixture was evaporated, extracted with EtOAc and the extract was dried and evaporated. The crude concentrate was purified by flash silica gel chromatography with 2:8 Hexanes:EtOAc, then EtOAc to elute the product. The fractions were concentrated to give pure 5′-DMT-3′-TBDMS-2′-arabinouridine as a yellow solid (100 g, 69.5%). The product identity was confirmed by ¹H NMR (300 MHz, DMSO-d₆, δ ppm) 11.39 (1H, d, J=2.2, NH), 7.61 (1H, d), 7.4 (9H, m), 6.95 (4H, m), 6.11 (1H, d, J=5), 5.81 (1H, d, J=5), 5.43 (1H, dd, J=8, 2.2), 4.18 (1H, m), 4.11 (1H, m), 3.86 (1H, m), 3.8 (6H, s), 3.4 (2H, m), 3.26 (1H, m), 0.843 (9H, s), 0.109 (3H, s), 0.024 (3H, s).

The 5′-DMT-3′-TBDMS-2′-arabinouridine (45 g, 69 mmol) was dissolved in pyridine (1000 mL) and to it was added 4-dimethylaminopyridine (DMAP, 1 g), acetic anhydride (Ac₂O, 12 ml, 96.6 mmol, 1.4 eq) and stirred at room temperature for 2 h. TLC (SiO₂, 3:7 Hexanes:EtOAc) showed complete reaction and then the reaction mixture was evaporated under reduced pressure and the residue coevaporated with toluene (200 mL). The residue was dissolved with dichloromethane and extracted with water and the organic layer was dried over Na₂SO₄ and evaporated to a concentrate. The crude product was purified by flash column chromatography with 3:7 Hexanes:EtOAc to give 5′-DMT-3′-TBDMS-2′acetyl-2′-arabinouridine, 4, (45 g, 93%) as a solid. The product identity was confirmed by ¹H NMR (300 MHz, DMSO-d₆, δ ppm) 11.4 (1H, d, J=2), 7.57 (1H, d, J=8), 7.33 (9H, m), 6.92 (4H, m), 6.23 (1H, d, J=5.5), 5.47 (1H, dd, J=8, 2.2), 5.17 (1H, t, J=53), 4.345 (1H, t, J=5.6), 4.0 (1H, m), 3.75 (6H, s), 3.35 (1H, m), 3.25 (1H, m), 2.25 (1H, m), 1.9 (3H, s), 0.77 (9H, s), 0 (3H, s), −0.84 (3H, s).

### Example 3

### Synthesis of Protected-Arabinocytidine 5

The fully protected ara-U, 4 is then aminated by a two-step process, triazolide formation and amination. The amination with ammonium hydroxide simultaneous deprotects the 2′-acetylyl to give 5. The 5′-DMT-3′-TBDMS-2′acetyl-2′-arabinouridinc, 4, (45 g, 64 mmol) was dissolved in acetonitrile (1500 mL) and to it were added 1,2,4-Triazole (72 g, 1.02 mol, 16 eq.) and triethylamine (143.3 mL, 103.2 g, 1.02 mol, 16 eq). To the reaction mixture cooled to 0° C. was added POCl₃ (24 mL, 39.3 g, 256 mmol, 4 eq) dropwise. After the addition was complete the reaction was stirred at room temperature until no starting material was observed by TLC (SiO₂, 2:3 Hexanes:EtOAc). The reaction mixture was filtered, washed with acetonitrile (1000 mL) and poured into a 2 gallon Parr reactor. To this was added concentrated ammonium hydroxide (500 mL), sealed and heated to 50° C. for 5 hours. After the reaction was cooled to room temperature, the acetonitrile was evaporated and the residue dissolved in EtOAc and extracted with water. The crude extract was dried with Na₂SO₄ and concentrated and the concentrate purified by flash silica gel chromatography with EtOAc as eluent. The pure fractions were evaporated to give 5′-DMT-3′-TBDMS-2′-arabinocytidine, 5 (34 g, 80%) as a solid. The product identity was confirmed by ¹H NMR (300 MHz, DMSO-d₆, δ ppm) 7.75 (1H, d, J=7.5), 7.42-7.2 (9H, m), 7.19 (2H, b), 6.87 (4H, m), 6.12 (1H, d, J=4.7), 5.62 (1H, d, J=7.5), 5.61 (1h, d, J=3), 4.1 (1H, m), 4.0 (1H, m), 3.78 (1H, m), 3.72 (6H, s), 3.4 (1H, m), 3.2 (1H, m), 0.8 (9H, s), 0.05 (3H, s), −0.026 (3H, s).

### Example 4

### Synthesis of Arabinoocytidine Phosphoramidite, 6

The 5′-DMT-3′-TBDMS-2′-arabinocytidine, 5 (34 g, 51 mmol) was dissolved in pyridine (300 mL) and to it was added DMAP (622 mg, 5.1 mmol), acetic anhydride (10.6 mL, 112.2 mmol, 2.2 eq). The mixture was stirred at room temperature for 2 h. TLC (SiO₂, 3:7 Hexanes:EtOAc) showed complete reaction and the reaction mixture was evaporated under reduced pressure and the residue dissolved with dichlormethane (300 mL). The crude product was purified by flash column chromatography with EtOAc to give 5′-DMT-3′-TBDMS-N⁴,2′-diacetyl-2′-arabinocytidine, (35 g, 92.%) as a solid. The product identity was confirmed by ¹H NMR (300 MHz, DMSO-d₆, δ ppm) 8.8 (1H, d, J=7.5), 7.4-7.2 (9H, m), 7.2 (1H, d, J=7.5), 6.8 (4H, m), 6.28 (1H, d, J=4.8), 5.03 (1H, m), 4.37 (1H, m), 4.16 (1H, m), 3.7 (6H, s), 3.46 (1H, m), 3.35 (1H, m), 2.7 (3H, s, H₂O), 1.9 (5H, m), 1.7 (3H, s), 0.8 (9H, s), 0.08 (3H, s), −0.03 (3H, s).

The 5′-DMT-3′-TBDMS-N⁴,2′-diacetyl-2′-arabinocytidine, (35 g, 47 mmol) was dissolved in dichloromethane (20 mL) and treated with 1.0 M tetrabutylammonium fluoride solution in tetrahydrofuran (TBAF in THF, 61 mL, 61 mmol, 1.3 eq). TLC (SiO₂, 1:9 MeOH:EtOAc) showed complete reaction and the reaction mixture was evaporated under reduced pressure and the residue dissolved with dichloromethane (100 mL). The crude product was purified by, flash column chromatography with EtOAc to give 5′-DMT-N⁴,2′-diacetyl-2′-arabinocytidine, (20 g, 68%) as a solid. The product identity was confirmed by ¹H NMR (300 MHz, DMSO-d₆, δ ppm) 10.9 (1H, s), 7.87 (1H, d, J=7.5) 7.34 (8H, m), 7.12 (1H, d, J=7.5), 6.92 (4H, m), 6.2 (1H, d, J=4.8), 5.95 (1H, d, J=4.6), 5.25 (1H, m), 4.13 (2H, m), 3.746 (6H, s), 3.35 (3H, s, H₂O), 3.3 (1H, m), 2.1 (3H, s), 1.74 (3H, s).

The free 3′-hydroxyl is then phosphitylated as follows. 5′-DMT-N⁴,2′-diacetyl-2′-arabinocytidine, (20 g, 31.7 mmol) was dissolved in dichloromethane (200 mL) and treated with triethylamine (TEA, 21.7 mL, 155 mmol, 5 eq.). To this solution was added chloro-2-cyanoethyl-N,N-diisopropylamino phosphoramidite (9.2 mL, 41.21 mmol, 1.3 eq) and stirred for 30 min at room temperature. TLC (SiO₂, 1:9 MeOH:EtOAc) showed complete reaction and the reaction mixture was extracted with aq. NaHCO₃. The organic layer was separated, dried and evaporated under reduced pressure. The residue dissolved with EtOAc (100 mL) and purified by flash column chromatography (1500 mL) to give pure phosphoramidite 6, (17 g, 64.4%) as a solid. The product identity was confirmed by ³¹P NMR (121.4 MHz, DMSO-d₆, δ ppm) 150.964, 150.432.

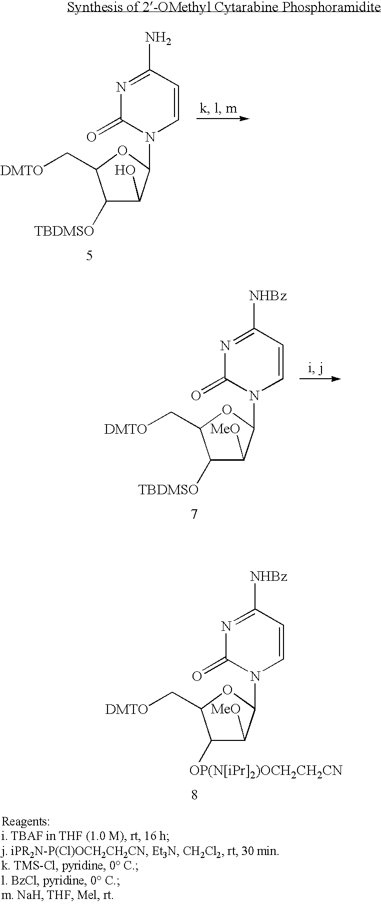

### Example 5

### Synthesis of 2′O-methyl cytarabine derivative 7

The 5′-DMT-3′-TBDMS-2′-arabinocytidine, 5 (15 g, 22.73 mmol, 1.0 eq) was dissolved in pyridine (100 mL) and cooled to 0° C. To this solution was added chlorotrimethylsilane (TMSCl, 9 mL, 68.2 mmol, 3 eq) dropwise and stirred for two hours at 0° C. To the reaction mixture was then added benzoyl chloride (5 mL, 40 mmol, 1.8 eq) and then stirred for 2 h at room temperature. The reaction mixture was quenched with concentrated NH₄OH (60 mL) and stirred for 30 min. The reaction mixture was then evaporated to dryness and the residue coevaporated with toluene. The residue was dissolved in dichloromethane and extracted with water. The crude concentrate was purified by flash silica gel column chromatography and eluted with 3:7 Hexanes:EtOAc to give pure 5′-DMT-3′-TBDMS-N⁴-Benzoyl-2′-arabinocytidine (13 g, 74.8%). The product was taken to the next step for 2′O-Methylation.

The 5′-DMT-3′-TBDMS-N⁴-Benzoyl-2′-arabinocytidine (8 g, 10.5 mmol) was dissolved in dry THF (150 mL) and to this was added sodium hydride (60%, 1.2 g, 30 mmol, 3 eq) at room temperature. The reaction was stirred for 1 h at room temperature and then iodomethane (1.12 mL, 18 mmol, 1.8 eq) was added. TLC (SiO₂, 3:7 Hexanes:EtOAc) showed incomplete reaction and additional aliquots of iodomethane was added to complete reaction. Two distinct products [“A”—higher Rf; “B”—lower Rf] are observed by TLC (syn and anti conformations of the base with restricted rotation). The products syn- and anti-5′-DMT-3′-TBDMS-N⁴-Benzoyl-2′-O-Methyl-2′-arabinocytidine, 7, were purified by flash silica gel column chromatography to give a combined 6.8 g (83%). The product identity was confirmed by ¹H NMR (600 MHz, DMSO-d₆, δ ppm) [Compound “A”] 12.92 (1H, b, NH), 8.12 (1H, d, J=7.5), 7.98 (2H, m), 7.6 (1H, m), 7.5 (2H, m), 7.4-7.2 (9H, m), 7.18 (1H, m), 6.9 (4H, m), 6.3 (1H, d, J=5.6), 4.2 (1H, t, J=5.6), 3.94 (1H, t, J=5.6), 3.89 (1H, m), 3.73 (6H, s), 3.3 (1H, m), 3.17 (3H, s), 0.76 (9H, s), 0.03 (3H, s), −0.07 (3H, s). [Compound “B”] 8.0 (2H, m), 7.66 (1H, d, J=7.5), 7.6 (1H, m), 7.5 (2H, m), 7.42-7.2 (9H, m), 7.06 (1H, d, J=7.5), 6.9 (4H, m), 6.01 (1H, d), 4.3 (1H, m), 4.27 (1H, m), 3.7 (6H, s), 3.61 (1H, m), 3.29 (3H, s), 3.2 (2H, m).

### Example 6

### Synthesis of 2′O-methyl cytarabine phosphoramidite, 8

The 5′-DMT-3′-TBDMS-N⁴-Benzoyl-2′-O-Methyl-2′-arabinocytidine (1.2 g, 1.54 mmol, 1.0 eq) was dissolved in THF (40 mL) and to it was added 1.0M TBAF in THF (5 mL) and stirred at room temperature. TLC (SiO₂, 2:8 Hexanes:EtOAc) showed complete reaction after 15 mins. The THF was evaporated and the residue dissolved in 2:8 Hexanes:EtOAc and separated by flash silica gel chromatography. The desired product with free 3′-hydroxyl group, 5′-DMT-N⁴-Benzoyl-2′-O-Methyl-2′-arabinocytidine was obtained as a solid (700 mg, 76%). The product identity was confirmed by ¹H NMR (300 MHz, DMSO-d₆, δ ppm) 11.28 (1H, bm), 8.0 (3H, m), 7.63 (1H, m), 7.53 (2H, m), 7.3 (10H, m), 6.92 (4H, d), 6.26 (1H, d), 5.74 (1H, d), 4.14 (1H, m), 3.98 (1H, m), 3.92 (1H, m), 3.75 (6H, s), 3.35 (3H, s), 3.29 (2H, m), 3.18 (3H, s).

The 5′-DMT-N⁴-Benzoyl-2′-O-Methyl-2′-arabinocytidine (2.0 g, 3.34 mmol, 1.0 eq) was dissolved in dichloromethane (60 mL) and to it was added triethylamine (2.3 mL, 16.5 mmol, 5 eq.) at room temperature. To this solution was added chloro-2-cyanoethyl-N,N-diisopropylamino phosphoramidite (1.34 mL, 1.42 g, 6.01 mmol, 1.8 eq). TLC (SiO₂, 1:9 MeOH:EtOAc) showed complete reaction after 30 mins. The reaction mixture was extracted with aq. NaHCO₃ and the organic layer dried over Na₂SO₄. The crude concentrate of the product was charged on a flash silica gel column and eluted with EtOAc followed by 1:9 MeOH:EtOAc to give the product, 5′-DMT-N⁴-Benzoyl-2′-O-Methyl-2′-arabinocytidine-3′-cyanoethyl-N,N-diisopropyl amino phosphoramidite, 8 as a foamy solid (2.15 g, 73%). The product identity was confirmed by ³¹P NMR (121.4 MHz, DMSO-d₆, δ ppm) 150.217, 150.161

Examples 7-12 describe the synthesis and purification of oligonucleotides (oligos 1-7) of the invention. All oligonucleotides were synthesized by Hybridon (Cambridge, Mass.) and/or Trilink Biotechnologies (San Diego, Calif.) using standard phosphoramidite chemistry. Synthesis and Properties of Oligonucleotides, by M. Ikehara, E. Ohtsuka, S. Uesugi, T. Tanaka, 283-367, in Chemistry of Nucleosides and Nucleotides, Vol. 1, Ed. Leroy B. Townsend, 1988. Synthesis of Oligonucleotide Phosphorothioates, G. Beaton, D. Dellinger, W. S. Marshall, M. H. Caruthers, 109-135 in Oligonucleotides and Analogues: A Practical Approach, Ed. F. Eckstein, IRL Press at Oxford University Press, NY, 1991; Oligophosphorothioates, Gerald Zon, 165-190, in Protocols for Oligonucleotides and Analogs: Synthesis and Preparation, Ed. Sudhir Agrawal, 1994. Also see V. T. Ravikuman et al, Nucleosides & Nucleotides, 14 (6), 1219-1226, 1995: M. Andrade et al., Medicinal Chemistry Letters 4 (16) 2017-2022, 1994; A. A. Padmapriya et al, Antisense Research & Development, 4, 185-189, 1994.

The first nucleoside attached to the solid support is deoxycytidine. This choice was made to simplify the oligonucleotide synthesis. The first nucleoside on the unbound and deprotected oligonucleotides are easily cleaved and do not significantly impact the observations in in vitro experiments. Also in the analysis of the cleavage products the single deoxycytidine monophosphate (dCMP) serves as an internal standard. For studies in biological systems, the particular starting nucleoside may be loaded on the solid support.

### Example 7

### Synthesis and purification of (cytarabine-pO)₁₅-dC (Oligo 1)

The oligonucleotide was synthesized on Perseptive's Expedite 8909 DNA synthesizer using 1 μmole scale standard protocol. After the oligo was synthesized, it was deprotected with 1 ml of concentrated ammonium hydroxide at 55° C. for 18 hours. It was evaporated and purified with 20% polyacrylamide gel. After the gel purification, the oligo was desalted with C18 cartridge. Yields: 5 OD

### Example 8

### Synthesis and purification of ([cytarabine-pO]₃-[2′O-methylcytidine-pO]₂)₃ (Oligo 2)

The oligonucleotide was synthesized on Perseptive's Expedite 8909 DNA synthesizer using 1 μmole scale standard protocol. After the oligo was synthesized, it was deprotected with 1 ml of concentrated ammonium hydroxide at 55° C. for 18 hours. It was evaporated and purified with 20% polyacrylamide gel. After the gel purification, the oligo was desalted with C18 cartridge.

### Example 9

### Synthesis and purification of ([cytarabine-pO]₃-[2′O-methylcytarabine-pO]₂)₃-dC (Oligo 3)

The oligonucleotide was synthesized on Perseptive's Expedite 8909 DNA synthesizer using 1 μmole scale standard protocol. After the oligo was synthesized, it was deprotected with 1 ml of concentrated ammonium hydroxide at 55° C. for 18 hours. It was evaporated and purified with 20% polyacrylamide gel. After the gel purification, the oligo was desalted with C18 cartridge. Yields: 8 OD

### Example 10

### Synthesis and purification of ([cytarabine-pO]₃-[2′O-methylcytidine-pS]₂)₃-dC (Oligo 4)

The oligonucleotide was synthesized on Perseptive's Expedite 8909 DNA synthesizer using 1 μmole scale standard protocol. After the oligo was synthesized, it was deprotected with 1 ml of concentrated ammonium hydroxide at 55° C. for 18 hours. It was evaporated and purified with 20% polyacrylamide gel. After the gel purification, the oligo was desalted with C18 cartridge. Yields: 8 OD

### Example 11

### Synthesis and purification of [2′O-methylcytarabine-pO]₁₅-dC (Oligo 5)

The oligonucleotide was synthesized on Perseptive's Expedite 8909 DNA synthesizer using 1 μmole scale standard protocol. After the oligo was synthesized, it was deprotected with 1 ml of concentrated ammonium hydroxide at 55° C. for 18 hours. It was evaporated and purified with 20% polyacrylamide gel. After the gel purification, the oligo was desalted with C18 cartridge. Yields: 10 OD

### Example 12

### Synthesis and purification of [dC-pO]₁₄-dC-OH (Oligo 6)

The oligonucleotide was synthesized on Perseptive's Expedite 8909 DNA synthesizer using 1 μmole scale standard protocol. After the oligo was synthesized, it was deprotected with 1 ml of concentrated ammonium hydroxide at 55° C. for 18 hours. It was evaporated and purified with 20% polyacrylamide gel. After the gel purification, the oligo was desalted with C18 cartridge. Yields: 5 OD

Examples 13-22 describe cleavage of the Oligos 1-7. All cleavage experiments were performed at room temperature. The concentrations of the nucleases were reduced to slow down the rates of cleavage for the homopolymers. See S. Agrawal, in Delivery Strategies for Antisense Oligonucleotide Therapeutics, 275, 462-473, Ed. S. Akhtar, CAC Press, 1995. The Capillary Gel Electrophoresis equipment used for the studies were the Beckman PAC/E 2200 system with Beckman Coulter E-CAP DNA capillary (#477477, 100 μM ID, 65 cm [27 cm effective length] loaded with Beckman Coulter E-CAP ssDNA 100-R gel and run using the Tris-Borate-Urea buffer prepared according to Beckman procedures. The CGE running conditions were as follows. The injection was done for 2 seconds at 10 kV followed by electro-osmotic flow with buffer for 30-60 min as needed at 10 kV. The cleavage reactions were monitored in most cases after 1, 2, 4, 5, 7, 10, 12, 15, 17 and 20 hrs. Some reactions were monitored after 5, 10 minutes and others after 72 and 100 hrs. The SVPDE is obtained from two different species of snakes, crotalus adamanteus and crotalus durissus. It has been observed that there is no variation in the cleavage experiments as a result of this change.

Preparation of Enzyme [Snake Venom PhosphoDiEsterase, SVPDE] Stock Solution: The dry lyophilized powder of the enzyme (0.5 units [0.33 units/mg], Phosphodiesterase I Type II; crotalus adamanteus; Sigma Product number P6877, Lot#71H97511) was dissolved in 1 mL of Tris buffer (Reliable Biopharmaceutical) to make the stock solution.

### Example 13

### Cleavage of Oligo 1

To 0.5 OD of the oligo 1 from its stock solution in a vial placed in the sample holder of the Beckman capillary gel electrophoresis (CGE) at room temperature was pipetted 0.00125 units of enzyme stock solution. The digestion of the oligonucleotide was then monitored by CGE at various time points. The digestion result was analyzed by monitoring the relative ratios of the two degradation products, dCMP and araCMP. As the cleavage progressed the ratio of araCMP to dCMP increased to 13 which is close to the theoretical maximum of 15. It is estimated that more than 50% was cleaved after 4 h.

### Example 14

### Cleavage of Oligo 2

To 0.5 OD of the oligo 2 from its stock solution in a vial placed in the sample holder of the Beckman capillary gel electrophoresis (CGE) at room temperature was pipetted 0.00125 units of enzyme stock solution. The digestion of the oligonucleotide was then monitored by CGE at various time points. The digestion result was analyzed by monitoring the relative ratios of the three degradation products, dCMP, 2′O-MeCMP and araCMP. As the cleavage progressed the ratio of araCMP to dCMP increased to 6, which is close to the theoretical maximum of 9. Similarly the ratio of 2′O-MeCMP to dCMP increased to 5, which is close to the theoretical maximum of 6. It was estimated that more than 50% of the full length oligo was cleaved after 6 hours.

### Example 15

### Cleavage of Oligo 3

To 0.5 OD of the oligo 3 from its stock solution in a vial placed in the sample holder of the Beckman capillary gel electrophoresis (CGE) at room temperature was pipetted 0.00125 units of enzyme stock solution. The digestion of the oligonucleotide was then monitored by CGE at various time points. After digestion for 72 h there was no significant cleavage or degradation of the oligonucleotide observed by CGE.

### Example 16

### Cleavage of Oligo 3 (with Higher Enzyme Concentration)

To 0.5 OD of the oligo 3 from its stock solution in a vial placed in the sample holder of the Beckman capillary gel electrophoresis (CGE) at room temperature was pipetted 0.25 units of enzyme stock solution. The digestion of the oligonucleotide was then monitored by CGE at various time points. After digestion for 15 h there was complete degradation of the oligonucleotide observed by CGE.

### Example 17

### Cleavage of Oligo 4

To 0.5 OD of the oligo 4 from its stock solution in a vial placed in the sample holder of the Beckman capillary gel electrophoresis (CGE) at room temperature was pipetted 0.00125 units of enzyme stock solution. The digestion of the oligonucleotide was then monitored by CGE at various time points. After 4 h there was very little degradation of the oligonucleotide observed by CGE. After 67 h, most of the full length oligonucleotide was degraded to monomers and shorter oligomers.

### Example 18

### Cleavage of Oligo 5

To 0.5 OD of the oligo 5 from its stock solution in a vial placed in the sample holder of the Beckman capillary gel electrophoresis (CGE) at room temperature was pipetted 0.00125 units of enzyme stock solution. The digestion of the oligonucleotide was then monitored by CGE at various time points. After digestion for 5 h there was no degradation of the oligonucleotide.

### Example 19

### Cleavage of Oligo 5 (with 40× Enzyme Concentration)

To 0.5 OD of the oligo 5 from its stock solution in a vial placed in the sample holder of the Beckman capillary gel electrophoresis (CGE) at room temperature was pipetted 0.05 units of enzyme stock solution. The digestion of the oligonucleotide was then monitored by CGE at various time points. After digestion for 24 h there was low levels of degradation products of the oligonucleotide observed by CGE.

### Example 20

### Cleavage of Oligo 5 (with 100× Enzyme Concentration)

To 0.5 OD of the oligo 5 from its stock solution in a vial placed in the sample holder of the Beckman capillary gel electrophoresis (CGE) at room temperature was pipetted 0.125 units of enzyme stock solution. The digestion of the oligonucleotide was then monitored by CGE at various time points. After digestion for 3 h degradation was observed and after 20 h more than 80% of the oligonucleotide was degraded.

### Example 21

### Cleavage of Oligo 6

To 0.5 OD of the oligo 6 (poly-deoxycytidine 15 mer, Hybridon, Inc) in a vial and placed in the sample holder of the Beckman capillary gel electrophoresis (CGE) at room temperature was pipetted 0.5 μl of snake phosphodiesterase from crotalus durissus (Boehringer Mannheim) in water (total volume 25 μl). The digestion of the oligonucleotide was then monitored by CGE at regular intervals. After 30 minutes, a series of shorter oligomers due to partial cleavage was observed. After 2 hours no full length oligonucleotide was observed and the largest peaks were the products, dCMP and dimers.

To 0.5 OD of the oligo 6 (poly-deoxycytidine 15 mer, Hybridon, Inc), from its stock solution in a vial and placed in the sample holder of the Beckman capillary gel electrophoresis (CGE) at room temperature was pipetted 0.00125 units of enzyme stock solution. The digestion of the oligonucleotide was then monitored by CGE at 10 minutes, 1, 2 and 17 h. After 10 minutes, a series of shorter oligomers due to partial cleavage was observed. The digestion was almost complete (more than 90%) after 5 minutes. The digestion products (dCMP) appear earlier (10-13 minutes) than the full oligonucleotide. After 2 hours the amount of full length oligonucleotide was minimal and the largest peaks were the products, dCMP and other oligomers.

### Example 22

### Cleavage of Oligo 7 (Polycylidilic Acid)

Oligo 7 is polycylidilic acid, a polymeric ribocytidine with phosphodiester linkages. To 0.5 OD of the oligo 7 (purchased from Sigma Chemical, Cat#81306), from its stock solution in a vial and placed in the sample holder of the Beckman capillary gel electrophoresis (CGE) at room temperature was pipetted 0.00125 units of enzyme stock solution. The digestion of the oligonucleotide was then monitored by CGE at 5 minutes, 1, 2 and 17 h. The digestion was almost complete (more than 90%) after 5 minutes. The digestion products appear earlier (10-13 minutes) than the full oligonucleotide.

### Example 23

### Analysis of Oligonucleotide Degradation

After degradation of the oligonucleotides as described in Examples 13-22, the resulting oligonucleotide was analyzed, using the single deoxycytidine monophosphate (dCMP) as an internal standard. The identity of the cleavage products was established by coinjection of standards of the cleavage products. The standards used were araCMP (cytarabine monophosphate obtained from Sigma), CMP (cytidine monophosphate obtained from Sigma) and dCMP (deoxycytidine monophosphate, obtained from Reliable Biopharmaceutical). The electropherograms showed increased absorbances of the peaks corresponding to the standards relative to the cleavage reaction mixture.

The degradation or cleavage study results are described below.

Oligo 7, polycytidilic acid (polymeric ribocytidine with phosphodiester linkages) was treated with the enzyme solution and complete degradation of the oligonucleotide to the monomers was observed in approximately 10 minutes.

Further, when [dC-pO]₁₄-dC-OH, (Oligo 6, a 15-mer deoxycytidine with phosphodiester linkages) was treated with the enzyme solution, complete degradation of the oligonucleotide to the monomers was observed in less than 2 hours.

Under similar conditions the (cytarabine-pO)₁₅-dC, Oligo 1, showed minor cleavage after 1 hr, suggesting that the cleavage was slower than Oligos 7 and 6. After 2 h the oligonucleotide was degraded to monomers (˜20%) and oligomers of smaller lengths (˜50%). It was estimated that greater than 50% of the full length oligo was cleaved after 4 hours. The extent of degradation was also determined by the relative ratios of the two degradation products, dCMP and araCMP. As the cleavage of the oligonucleotide progressed the ratio of araCMP to dCMP increased from 3 to 13, which is close to the theoretical maximum of 15. These experiments established that oligonucleotide prodrugs of the prior art are cleaved rapidly in the presence of nucleases.

Oligo 2, with a mixed cytarabine phosphodiester and 2′O-methyl-C-phosphodiester backbone based speed bump, showed no cleavage after 4 hrs but showed significant cleavage after 7 hours. It was estimated that more than 50% of the full length oligo was cleaved after 6 hours. This suggests that the cleavage of the oligonucleotide can be controlled by the incorporation of a modified nucleoside at specific locations on a cytarabine oligonucleotide. The extent of degradation was also determined by the relative ratios of the three degradation products, dCMP, 2′O-MeCMP and araCMP. As the cleavage progressed the ratio of araCMP to dCMP increased to 6, which is close to the theoretical maximum of 9. Similarly, the ratio of 2′O-MeCMP to dCMP increased to 5, which is close to the theoretical maximum of 6.

Oligo 4, with a mixed cytarabine phosphodiester and 2′O-methylcytidine-phosphorothioate backbone based speed bump, shows no cleavage after 4 hrs but shows some cleavage after 8 hours. After 67 hours, most of the full length oligonucleotide was degraded to monomers and shorter oligomers. This suggests that the cleavage of the oligonucleotide can be finely controlled, in this case delayed longer by the incorporation of a phosphorothioate backbone in addition to the 2′O-methyl cytidine nucleosides at specific locations on a cytarabine oligonucleotide.

Oligo 3, with the mixed cytarabine and 2′O-methyl-cytarabine all phosphodiester backbone based speed bump shows no cleavage or degradation of the oligonucleotide after 4, 8 and 72 hours as observed by CGE. The 2′O-methyl araC nucleoside has been identified as a potential speed bump molecule. This suggests that the cleavage of the oligonucleotide can be stopped, that is nuclease resistance achieved by the incorporation of these nucleosides in specific locations on a cytarabine oligonucleotide.

When the enzyme concentration was increased 500 fold to 0.25 units of the enzyme stock solution and the digestion of the oligonucleotide was then monitored by CGE at various time points, there was complete degradation of the oligonucleotide after 15 hours.

Oligo 5, a 15-mer of 2′O-methylcytarabine with phosphodiester linkages under the same cleavage or degradation conditions is not degraded at all by nucleases after 5 and 20 hours.

Then, the enzyme concentration was increased 40 fold to 0.05 units of the enzyme stock solution and the digestion of the oligonucleotide (Oligo 5) was then monitored by CGE at various time points. After digestion for 24 hours, low levels of degradation products of the oligonucleotide were observed by CGE.

The enzyme concentration was further increased 100 fold to 0.125 units of the enzyme stock solution and the digestion of Oligo 5 was monitored at various time points. After 3 hours, some degradation was observed. After 20 hours more than 80% of the oligonucleotide was degraded.

Comparing the cleavage results of the 2′O-methyl cytarabine homopolymer with that of the cytarabine homopolymer suggests that the nuclease resistance is derived solely from the alkylation of the arabino sugar hydroxyl. It is expected that the abasic 2′O-alkylated arabinonucleoside, when incorporated into oligonucleotides, may confer the same nuclease resistance.

### Example 24

### Synthesis of poly-2-chloro-2′-deoxyadenosine via phosphoramidite

Cladribine is N⁶-acylated using published methods and is then converted to its phosphoramidite by conventional methods. Preparation of Protected Deoxyribonucleosides, R. A. Jones 23-34, in Oligonucleotide Synthesis: A Practical Approach, Ed. M. J. Gait, IRL Press at Oxford University Press, NY, 1984 and Oligodeoxyribonucleotide Synthesis: Phosphoramidite Approach, S. L. Beaucage, 33-61, in Protocols for Oligonucleotides and Analogs: Synthesis and Preparation, Ed. Sudhir Agrawal, 1994. Polycladribine, with varying phosphodiester and phosphorothioate linkages is prepared using a DNA synthesizer and the 2-chlorodeoxyadenosine phosphoramidite. The crude product is purified by HPLC and analyzed by NMR, MS or similar analytical methods commonly used by those of ordinary skill in the art.

### Example 25

### Synthesis of poly-2-chloro-2′-deoxyadenosine using H-phosphonate

Cladribine is converted to its H-phosphonate and then polymerized as per published methods. See Oligonucleotide Synthesis: H-Phosphonate Approach, Brian C. Froehler, 63-80 in Protocols for Oligonucleotides and Analogs: Synthesis and Preparation, Ed. Sudhir Agrawal, 1994.

The crude product is purified by HPLC and analyzed by, NMR, MS or similar analytical methods commonly used by those of ordinary skill in the art.

### Example 26

### Synthesis of poly-2-chloro-2′-deoxyadenosine via triphosphates

Cladribine is phosphorylated by addition of POCl₃ from published methods. The resulting 2-chloro analog of DMP is converted to the corresponding triphosphate using methods taught by Bogachev, for example in V S Bogachev; Bioorg. Khim. 1996, 22, 699-705. The Synthesis, Reactions and Properties of Nucleoside Mono-, Di-, Triphosphates, etc by D. W. Hutchinson, 81-160, in Chemistry of Nucleosides and Nucleotides, Vol. 2, Ed. Leroy B. Townsend, 1991. The resulting triphosphate is then polymerized enzymatically.

The crude product is purified by HPLC, and may be analyzed by NMR, MS, or any of the other analytical methods commonly used by those of ordinary skill in the art.

### Example 27

### Synthesis of ¹⁴C, ³H, ³⁵S Labeled Polymers

Oligonucleotides with ³⁵S and Tritium[³H] radiolabels can be purchased from Trilink Biotechnologies, San Diego, Calif.

Radiolabeled polymers are formed according to the methods taught by S. Agrawal et al., in Delivery Strategies for Antisense Oligonucleotide Therapeutics, 105, Ed. S. Akhtar, CAC Press, 1995; R. M. S. Crooke et al., J. Pharmacology and Experimental Therapeutics 275, 462-473, 1995; H, M. Sansorn et al, J. Labeled Compounds and Radiopharmaceuticals, 36, 15-31, 1995; L-F Tao et al, Antisense Research & Development 5, 123-129, 1995; R. Ahange et al, Biochem. Pharmacol., 49, 929-939, 1995.

### Example 28

### In Vitro Degradation with Enzymes

The polynucleotide (0.1-1.0 mM conc) is treated with snake venom phosphodiesterase, an exonuclease in an appropriate solvent medium for a specified time and the degradation products analyzed by capillary gel electrophoresis. If the polynucleotide is ³²P-labelled [hot], then the degradation products can be visualized using a gel and the products quantitated by autoradiography using a phosphoimager. See S. Agrawal, in Delivery Strategies for Antisense Oligonucleotide Therapeutics, 275, 462-473, Ed. S. Akhtar, CAC Press, 1995.

### Example 29

### In Vitro Degradation with Cell Extracts

The polynucleotide (0.1-1.0 mM conc) is treated with cell extracts from blended liver (a mixture of exo and endonucleases) in an appropriate solvent medium for a specified time and the degradation products analyzed by capillary gel electrophoresis. The polynucleotide may be ³²P-labelled [hot], and the degradation products can then be visualized using a gel and the products quantitated by autoradiography using a phosphoimager. See S. Agrawal, in Delivery Strategies for Antisense Oligonucleotide Therapeutics, 275, 462-473, Ed. S. Akhtar, CAC Press, 1995.

### Example 30

### Intercellular Degradation in Stability Assays

An intercellular degradation assay can be conducted, as taught by R. M. S. Crooke et al., J. Pharmacology and Experimental Therapeutics 269, 89-94, 1994.

### Example 31

### Pharmacokinetics In Vivo

Animals are fed a mixture of the unlabelled and tritium (3H) labeled oligonucleotide. After a predetermined period has passed the animal is euthanized and the liver is harvested, and the relevant extract is analyzed for the presence of the oligonucleotides. The tritium counts show the presence of the full length and degraded oligonucleotides and their relative concentrations. See P. A. Cossum et al., J. Pharmacology and Experimental Therapeutics 269, 89-94, 1994.

While the invention has been illustrated with respect to the production and use of particular compounds, it is apparent that variations and modifications of the invention can be made without departing from the spirit or scope of the invention.

What is claimed is:

1. A heteropolymeric compound comprising a chain of monomeric nucleosides, nucleoside analogs, abasic nucleosides, or heterocyclic derivatives thereof, wherein each of said nucleosides, nucleoside analogs, abasic nucleosides, or heterocyclic derivatives thereof is pharmaceutically active and said nucleosides, nucleoside analogs, abasic nucleosides or heterocyclic derivatives thereof are linked by a phosphodiester group comprising a 3′ or 5′ terminal moiety, phosphorothioate group, or H—, alkyl or alkenyl phosphonate group.

2. The compound of claim 1, wherein said nucleosides are selected from the group consisting of adenosine, 5-azacytidine, cladribine, cytarabine, doxifluridine, enocitabine, floxuridine, fludarabine, gemcitabine, pentostatin, brivudine, edoxudine, fiacitabine, fialuridine, ibacitabine, idoxuridine, ribavirin, trifluridine and vidarabine.

3. The compound of claim 1, wherein said nucleoside analogs are carbacylic analogs or L-nucleosides.

4. The compound of claim 1, wherein said nucleoside analogs are selected from the group consisting of acyclovir, valacyclovir, penciclovir, famciclovir, ganciclovir, cidofovir, adefovir, lobucavir and ribavirin.

5. The compound of claim 1, wherein said nucleobases are selected from the group consisting of mercaptopurine, thioguanine and azathioprine.

6. The compound of claim 1, wherein said chain comprises from 2 to 100 monomeric nucleoside, nucleoside analogs, abasic nucleosides or heterocyclic derivatives thereof.

7. The compound of claim 1, wherein at least one of said nucleosides, nucleoside analogs, abasic nucleosides or heterocyclic derivatives thereof are antiviral.

8. The compound of claim 1, wherein at least one of said nucleosides, nucleoside analogs, abasic nucleosides or heterocyclic derivatives thereof is pharmaceutically active against cancer.

9. The compound of claim 1, wherein at least one of said nucleosides, nucleoside analogs, abasic nucleosides or heterocyclic derivatives thereof are antimicrobial.

10. A method of treating a viral infection in a patient in need thereof, said method comprising administering an effective amount of a compound of claim 7.

11. A method of treating cancer in a patient in need thereof, said method comprising administering an effective amount of a compound of claim 8.

12. A method of treating a microbial infection in a patient in need thereof, said method comprising administering an effective amount of a compound of claim 9.

13. A pharmaceutical composition comprising a compound of claim 1 and a pharmaceutically acceptable carrier.

14. A heteropolymeric compound of general formula (I) 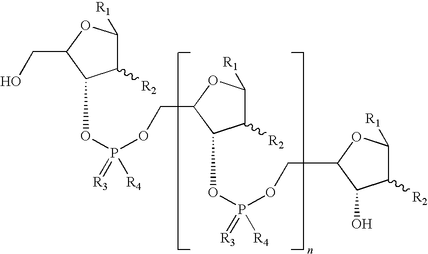
wherein R¹ is optionally present and if present is independently selected from the group consisting of a pharmaceutically active nucleoside, nucleoside analog or heterocyclic derivative thereof;
R² is present in the β or a face and is independently selected from the group consisting of hydrogen, O—R⁵, R⁵, N—R⁵R⁶, N₃, X, or S—R⁵; wherein R⁵ and R⁶ are independently selected from the group consisting of hydrogen, C₁-C₃₅ alkyl, C₂₋₃₅ alkenyl, C₃₋₃₅ cycloalkyl, C₁₋₃₅ alkoxy, C₁₋₃₅ alkylamino, C₂₋₃₅ ether, C₂₋₃₅ thioether, aryl, C₆₋₃₅ non-aromatic heterocyclic, or a heteroaryl;
X is Cl, Br, F, or I;
R³ is independently selected from the group consisting of O or S;
wherein when R³ is S, R⁴ is O⁻ and when R³ is O, R⁴ is selected from the group consisting of C₁₋₅ alkyl, C₁₋₅ alkenyl and O⁻;
wherein said alkyl, alkenyl, cycloalkyl, alkoxy, alkenyloxy, aryl, non-aromatic heterocyclic or heteroaryl are optionally substituted with one or more substituents selected from the group consisting of halogen, hydroxy, amino, acyloxy and carboxy.
n is an integer from 1-100;
or a pharmaceutically acceptable salt thereof.

15. The compound of claim 14, wherein R¹ is selected from the group consisting of 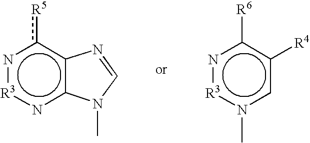
R³ is CH, C═O, C═S or NH₂⁻;
R⁴ is H, C₁₋₃₅ alkyl, C₂₋₃₅ alkenyl, C₃₋₃₅ cycloalkyl, C₁₋₃₅ alkoxy, C₁₋₃₅ alkenyloxy, C₁₋₃₅ alkylamino, C₂₋₃₅ ether, C₂₋₃₅ thioether, aryl, C₆₋₃₅ non-aromatic heterocyclic or heteroaryl;
R⁵ is O, S or NH₂; and
R⁶ is H, C═O, C═S, NH₂, NHR⁷, or SR⁷, wherein R⁷ is selected from the group consisting of C₁₋₃₅ alkyl, C₂₋₃₅ alkenyl, C₃₋₃₅ cycloalkyl, C₁₋₃₅ alkoxy, C₁₋₃₅ alkenyloxy, C₁₋₃₅ alkylamino, C₂₋₃₅ ether, C₂₋₃₅ thioether, aryl, C₃₋₃₅ non-aromatic heterocyclic or heteroaryl;
wherein said alkyl, alkenyl, alkenyloxy, cycloalkyl, alkoxy, aryl, non-aromatic heterocyclic and heteroaryl are optionally substituted with one or more substituents selected from the group consisting of halogen, hydroxy, amino, acyloxy and carboxy.

16. The compound of claim 14, wherein each R¹ is selected from the group consisting of nucleoside analogs.

17. The compound of claim 14, wherein said nucleoside analog is an acyclic, monocyclic or polycyclic moiety.

18. The compound of claim 14 wherein each R⁴ is O—.

19. The compound of claim 14, wherein R¹ is independently selected from the group consisting of optionally substituted adenine, guanine, cytosine, uracil and thymine or a heterocyclic base derivative thereof.

20. The compound of claim 14, wherein each R¹ is independently selected from the group consisting of adenine, cytosine, 2,6-diaminopurine, 2-chloroadenine, 6-mercaptopurine, thioguanine, 5-Fluorouracil and 2-Fluoroadenine.

21. A compound of general formula II 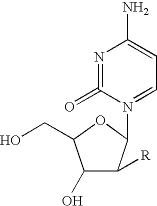
wherein R is selected from the group consisting of a C₁₋₃₅ alkyl, C₁₋₃₅ alkenyl, C₃₋₃₅ cycloalkyl, C₁₋₃₅ alkoxy, C₁₋₃₅ alkylamino, C₂₋₃₅ ether, C₂₋₃₅ thioether, C₂₋₃₅ alkenyloxy, aryl, C₆₋₃₅ non-aromatic heterocyclic, or heteroaryl;
wherein said alkyl, alkenyl, cycloalkyl, alkoxy, alkenyloxy, aryl, non-aromatic heterocyclic and heteroaryl are optionally substituted with one or more substituents selected from the group consisting of halogen, hydroxy, amino, acyloxy and carboxy.

22. The compound of claim 21, wherein R is —OCH₂CH₂OCH₃.

23. A compound of general formula III 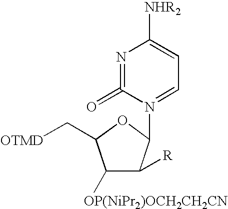
wherein R² is —C(O)R wherein R is independently selected from the group consisting of C₁₋₃₅ alkyl, C₃₋₃₅ cycloalkyl, C₁₋₃₅ alkoxy, C₁₋₃₅ alkylamino, C₂₋₃₅ ether, C₂₋₃₅ thioether, C₂₋₃₅ alkenyl, C₂₋₃₅ alkenyloxy, aryl, a C₆₋₃₅ non-aromatic heterocyclic, and heteroaryl;
wherein said alkyl, alkenyl, cycloalkyl, alkoxy, aryl, non-aromatic heterocyclic and heteroaryl are optionally substituted with one or more substituents selected from the group consisting of halogen, hydroxy, amino, acyloxy and carboxy.

24. The compound of claim 23, wherein R is alkoxy.

25. The compound of claim 23, wherein R is —OCH₂CH₂OCH₃.

26. The compound of claim 21, appended singly or as multimers or as groups of multimers to an oligonucleotide or analog at the 3′-, 5′- or at both termini.

27. The compound of claim 23, appended singly or as multimers or as groups of multimers to an oligonucleotide or analog at the 3′-, 5′- or at both termini.

28. A compound of general formula (IV) 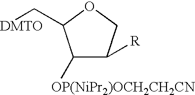
wherein R is selected from the group consisting of hydrogen, C₁₋₃₅ alkyl, C₂₋₃₅ alkenyl, C₃₋₃₅ cycloalkyl, C₁₋₃₅ alkoxy, C₂₋₃₅ alkenyloxy, C₁₋₃₅ alkylamino, C₂₋₃₅ ether, C₂₋₃₅ thioether, aryl C₆₋₃₅ non-aromatic heterocyclic, and heteroaryl.

29. The compound of claim 28, wherein R is methyl or ethyl.

30. The compound of claim 29, wherein R is methyl.

31. The compounds of claim 28 wherein R is OCH₂CH₂OCH₃.
